SEQUENCE LISTING (50 sequences)

SEQ ID NO: 1 - protein, 261 aa, Homo sapiens
  Prostate-specific antigen (PSA)
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 123 aa, Homo sapiens
  Prostate stem cell antigen (PSCA)
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 3 - protein, 750 aa, Homo sapiens
  Prostate specific membrane antigen (PSMA)
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 4 - protein, 339 aa, Homo sapiens
  Six-transmembrane epithelial antigen of the prostate (STEAP1)
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 5 - protein, 1255 aa, Homo sapiens [sequence omitted]

SEQ ID NO: 6 - protein, 1255 aa, Homo sapiens
  Mucin 1, cell surface associated protein (MUC1)
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 7 - protein, 519 aa, Homo sapiens
  Tyrosinase-related protein 2 (TRP-2)
MSPLWWGFLLSCLGCKILPGAQGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVLHSFTDAIFDEWMKRFNPPADAWPQELAPIGHNRMYNMVPFFPPVTNEELFLTSDQLGYSYAIDLPVSVEETPGWPTTLLVVMGTLVALVGLFVLLAFLQYRRLRKGYTPLMETHLSSKRYTEEA

SEQ ID NO: 8 - protein, 766 aa, Homo sapiens [sequence omitted]

SEQ ID NO: 9 - protein, 976 aa, Homo sapiens [sequence omitted]

SEQ ID NO: 10 - protein, 189 aa, Homo sapiens
  GTPase NRas
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 11 - protein, 309 aa, Homo sapiens
  Melanoma-associated antigen 1
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 12 - protein, 314 aa, Homo sapiens
  Melanoma-associated antigen 1
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 13 - protein, 180 aa, Homo sapiens
  NY-ESO-1 protein
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 14 - RNA, 3822 nt, SARS coronavirus
  mRNA encoding SARS-CoV-2 Spike (S) protein
auguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaa

SEQ ID NO: 15 - protein, 254 aa, SARS coronavirus
  SARS-CoV-2 Spike (S) protein
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 16 - protein, 609 aa, SARS coronavirus
  SARS-CoV-2 Spike (S) protein
CDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKL

SEQ ID NO: 17 - RNA, 1260 nt, SARS coronavirus
  mRNA encoding SARS-CoV-2 nucleocapsid (N) protein
augucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaa

SEQ ID NO: 18 - protein, 355 aa, SARS coronavirus
  SARS-CoV-2 nucleocapsid (N) protein
RITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEP

SEQ ID NO: 19 - RNA, 669 nt, SARS coronavirus
  mRNA encoding SARS-CoV-2 membrane (M) protein
auggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaa

SEQ ID NO: 20 - protein, 218 aa, SARS coronavirus
  SARS-CoV-2 membrane (M) protein
SNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLV

SEQ ID NO: 21 - protein, 195 aa, Influenza A virus
  Hemagglutinin (HA) protein of influenza A virus H5N1
NDQGSGYAADKESTQKAFNGITNKVNSVIEKMNTQFEAVGKEFSNLEKRLENLNKKMEDGFLDVWTYNAELLVLMENERTLDFHDSNVKNLYDKVRMQLRDNVKELGNGCFEFYHKCDNECMDSVKNGTYDYPKYEEESKLNRNEIKGVKLSSMGVYQILAIYATVAGSLSLAIMMAGISFWMCSNGSLQCRICI

SEQ ID NO: 22 - protein, 538 aa, Influenza A virus
  Hemagglutinin (HA) protein of influenza A virus H3N2
LCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 23 - protein, 538 aa, Influenza A virus; identical to SEQ ID NO: 22
  Hemagglutinin (HA) protein of influenza A virus H1N1

SEQ ID NO: 24 - protein, 540 aa, Influenza A virus
  Hemagglutinin (HA) protein of influenza A virus H7N9
ICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 25 - protein, 540 aa, Influenza A virus
  Hemagglutinin (HA) protein of influenza A virus H1N1
ICIGHQSTNSTETVDTLTETNVPVTHAKELLHTEHNGMLCATSLGHPLILDTCTIEGLVYGNPSCDLLLGGREWSYIVERSSAVNGTCYPGNVENLEELRTLFSSASSYQRIQIFPDTTWNVTYTGTSRACSGSFYRSMRWLTQKSGFYPVQDAQYTNNRGKSILFVWGIHHPPTYTEQTNLYIRNDTTTSVTTEDLNRTFKPVIGPRPLVNGLQGRIDYYWSVLKPGQTLRVRSNGNLIAPWYGHVLSGGSHGRILKTDLKGGNCVVQCQTEKGGLNSTLPFHNISKYAFGTCPKYVRVNSLKLAVGLRNVPARSSRGLFGAIAGFIEGGWPGLVAGWYGFQHSNDQGVGMAADRDSTQKAIDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYREESRLERQKIEGVKLESEGTYKILTIYSTVASSLVLAMGFAAFLFWAMSNGSCRCNICI

SEQ ID NO: 26 - protein, 545 aa, Influenza A virus
  Hemagglutinin (HA) protein of influenza A virus H2N2
ICIGYHANNSTEKVDTILERNVTVTHAKDILEKTHNGKLCKLNGIPPLELGDCSIAGWLLGNPECDRLLSVPEWSYIMEKENPRYSLCYPGSFNDYEELKHLLSSVKHFEKVKILPKDRWTQHTTTGGSWACAVSGKPSFFRNMVWLTRKGSNYPVAKGSYNNTSGEQMLIIWGVHHPNDEAEQRALYQNVGTYVSVATSTLYKRSIPEIAARPKVNGLGRRMEFSWTLLDMWDTINFESTGNLVAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFNGITNKVNSVIEKMNTQFEAVGKEFSNLEKRLENLNKKMEDGFLDVWTYNAELLVLMENERTLDFHDSNVKNLYDKVRMQLRDNVKELGNGCFEFYHKCDNECMDSVKNGTYDYPKYEEESKLNRNEIKGVKLSSMGVYQILAIYATVAGSLSLAIMMAGISFWMCSNGSLQCRICI

SEQ ID NO: 27 - protein, 567 aa, Influenza B virus
  Hemagglutinin (HA) protein
ICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 28 - protein, 381 aa, Influenza A virus
  Neuraminidase (NA) protein of influenza A virus H5N1
LAGNSSLCPISGWAVHSKDNGIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELNAPNYHYEECSCYPDAGEITCVCRDNWHGSNRPWVSFNQNLEYQIGYICSGVFGDNPRPNDGTGSCGPVSPNGAYGVKGFSFKYGNGVWIGRTKSTNSRSGFEMIWDPNGWTGTDSSFSVKQDIVAITDWSGYSGSFVQHPELTGLDCIRPCFWVELIRGRPKESTIWTSGSSISFCGVNSDTVGWSWPDDAELPF

SEQ ID NO: 29 - protein, 383 aa, Influenza A virus
  Neuraminidase (NA) protein of influenza A virus H1N1
VILTGNSSLCPIRGWAIYSKDNSIRIGSKGDVFVIREPFISCSHLECRTFFLTQGALLNDRHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPKDGTGSCGPVYVDGANGVKGFSYRYGNGVWIGRTKSHSSRHGFEMIWDPNGWTETDSKFSVRQDVVAMTDWSGYSGSFVQHPELTGLDCIRPCFWVELIRGRPKEKTIWTSASSISFCGVNSDTVDWSWPDGAELPF

SEQ ID NO: 30 - protein, 384 aa, Influenza A virus
  Neuraminidase (NA) protein of influenza A virus H3N2
EYRNWSKPQCDITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKKILRTQESECVCINGTCTVVMTDGSASGKADTKILFIEEGKIIHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKEETEVLWTSNSIVVFCGTSGTYGTGSWPDGADINL

SEQ ID NO: 31 - protein, 384 aa, Influenza A virus
  Neuraminidase (NA) protein of influenza A virus H7N9
FNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEY

SEQ ID NO: 32 - protein, 384 aa, Influenza A virus
  Neuraminidase (NA) protein of influenza A virus H9N2
EYKNWSKPQCQITGFAPFSKDNSIRLSAGGDIWVTREPYVSCGLGKCYQFALGQGTTLNNKHSNGTIHDRSPHRTLLMNELGVPFHLGTKQVCIAWSSSSCHDGKAWLHVCVTGDDRNATASIIYDGMLTDSIGSWSKNILRTQESECVCINGTCTVVMTDGSASGRADTKILFIREGKIVHIGPLSGSAQHVEECSCYPRYPEVRCVCRDNWKGSNRPVLYINVADYSVDSSYVCSGLVGDTPRNDDSSSSSNCRDPNNERGGPGVKGWAFDNGNDVWMGRTIKKDSRSGYETFRVVGGWTTANSKSQINRQVIVDSDNWSGYSGIFSVEGKTCINRCFYVELIRGRPQETRVWWTSNSIIVFCGTSGTYGTGSWPDGANINF

SEQ ID NO: 33 - protein, 384 aa, Influenza A virus
  Neuraminidase (NA) protein of influenza A virus H2N2
EYRNWSKPQCQITGFAPFSKDNSIRLSAGGDIWVTREPYVSCDPGKCYQFALGQGTTLDNKHSNDTIHDRIPHRTLLMNELGVPFHLGTRQVCVAWSSSSCHDGKAWLHVCVTGDDKNATASFIYDGRLMDSIGSWSQNILRTQESECVCINGTCTVVMTDGSASGRADTRILFIEEGKIVHISPLSGSAQHVEECSCYPRYPDVRCICRDNWKGSNRPVIDINMEDYSIDSSYVCSGLVGDTPRNDDRSSNSNCRNPNNERGNPGVKGWAFDNGDDVWMGRTISKDLRSGYETFKVIGGWSTPNSKSQINRQVIVDSNNWSGYSGIFSVEGKRCINRCFYVELIRGRQQETRVWWTSNSIVVFCGTSGTYGTGSWPDGANINF

SEQ ID NO: 34 - protein, 381 aa, Influenza A virus
  Neuraminidase (NA) protein of influenza A virus H1N1
LAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPF

SEQ ID NO: 35 - protein, 466 aa, Influenza B virus
  Neuraminidase (NA) protein
MLPSTVQTLTLLLTSGGVLLSLYVSASLSYLLYSDVLLKFSSTKTTAPTMSLECTNASNAQTVNHSATKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 36 - protein, 842 aa, Human immunodeficiency virus 1
  Envelope protein
MRVRGMQRNWQHLGKWGLLFLGMLIICKAADDLWVTIYYGVPVWKEANTTLFCASDAKSYEKEVHNVWATHACVPTDPNPQEVALNVTENFNMWENDMVEQMHKDIISLWDQSLKPCVKLTPLCVTLNCTNATTTNDTLSDQSSTLKEEPGAIQNCSFNMTTEVEDKKQKVHALFYRLDIEPISNNNSREEYRLITCNTSTITQACPKVSWDPIPIHYCAPAGYAILKCKDKRFNGTGPCRNVSTVQCTHGIRPVVSTQLLLNGSLSEGGIIIRSQNLSDNAKTIIVHLNESVQINCTRPNNNTRKSIRIGPGQSFYATGEIIGDIRKAHCNISGEQWNKTLDRVKAELKLHFNKTIQFNSSSGGDLEITMHSFNCRGEFFYCNTSLLFNNTVPNNGTITLPCRIKQFVNMWQEVGRAMYAAPIAGNITCNSNITGLLLTRDGGQSNNSDSETFRPGGGDMKDNWRSELYKYKVVEIEPLGVAPTRPKRPVVRRERRAVAIGAVFLGFLSAAGSTMGAASLTLTVQARQLLSGIVQQQNNLLQAIEAQQHMLQLTVWGIKQLQARVLAVERYLKDQQLLGLWGCSGKLICTTNVPWNSSWSNKSQDEIWNNMTWMQWEKEISNYSKTIYMLIEKSQSQQERNEQELLELDKWDSLWSWFDITNWLWYIKIFIMIVGGLIGLRIVFAVLSIVNRVRKGYSPLSLQTLIPAPTEPDRPEGIEEGGGEQGKDRSVRLVNGFLALVWDDLRNLCLFSYRHLRDFILIAARIVDRGLRRGWEALKYLGNIIQYWSQELKNSAISLFNTTAIVVAEGTDRVIEALQRAVRAVLNIPRRIRQRVERALI

SEQ ID NO: 37 - protein, 850 aa, Human immunodeficiency virus 2
  Envelope protein
MTSEKTQLLIAILLASTCLLYCKQYVTVFYGVPAWRNASIPLFCATKNRDTWGTIQCLPDNDDYQEIALNVTEAFDAWNNTVTEQAIDDVKRLFETSIKPCVKLTPLCVAMNCTNVTSTANTTITISSTNMIVNDSSPCASNDSCPGMGEEEMVQCNFSMTGLQRDKVKQYNETWYSRDVVCDQGENDTRRCYMNHCNTSVITESCDKHYWDDMRFRYCAPPGYALLRCNDTNYSGFMPNCSKVVAATCTRMMETQTSTWFGFNGTRAENRTYIYWHSKSNRTIISLNKSFNLSLHCKRPGNKTVVPITLMSGLVFHSQPINTRPRQAWCWFEGEWKEAMQEVKRAIAKHPRYTGTNDTGKINLTAPGRGSDPEVSYMWTNCRGEFLYCNMTWFLNWVENRTGIQRNYAPCHIRQIINTWHKVGKNVYLPPREGELVCNSTVTSLIADIDWIDKNGTNITFSAEVSELYRLELGDYKLVEITPIGFAPTTEKRYSPNHGRPKRGVFVLGFLGFLATAGSAMGAASLTLSAQSRTLLAGIVQQQQQLLDVVKRQQEMLRLTVWGTKNLQARVTAIEKYLGDQARLNSWGCAFRQVCHTTVPWVNDTLMPDWKNMTWQKWEEQIRYLEANISDSLEQAQIQQEKNTYELQKLNSWDVFTNWLDLTSWIKYIQYGVYIVVGIIALRIVIYVVQMLSRLRKGYRPVFSSPPGYIQQIHIHKGQEQPTREGTEEDVGDNGGDRSWPWPIAYLHFLIRLLIRLLITLYNSCRDLLSRIFQTLQPILRNLRDWLRIKTALLQYGCEWIQEAFQAAARTTGETLAGACRGLWRTLGRIGRGIFAVPRRIRQGAEIALL

SEQ ID NO: 38 - protein, 424 aa, Human papillomavirus
  Major Capsid L1 Protein
AVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTQVAVQPGDCPPLELINTVIQDGDMVDTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNGGSGGEDPLKKYTFWEVNLKEKFSADLDQFPLGRKFLLQL

SEQ ID NO: 39 - protein, 523 aa, Human papillomavirus
  Minor Capsid L2 Protein
MARARRTKRDSVTNIYRTCKAAGTCPPDVVNKVEQTTVADQILKYGSTGVFFGGLGIGTGRGTGGSTGYGPLGEGTSVRVGNTPTVIRPALVPEAIGPSELIPIDSVNPIDPSASSIIPLTESTGPDLLPGEIETIAEVHPAPDIPTVDTPVVTGGRNSNAVLEVADPSPPTRNRVSRTQYNNPAFQIISETTPSAGETSLSDQIVVQSFDGGQYIGGNPPPRSVVEIELQEIPSQYSFEIEEPTPPRQTSTPVRQAQQMASALRRALYNRRFTQQVQVEDPMFYSRPSRLVRFQFDNPVFEEEVTQVFERDLETIEEPPDRQFLDVQKLGRPTYAETPAGYIRVSRLGKRATIRTRSGTTIGGQVHFFRDISSIDTQPSIELQVLGEHSGDATIVQGPVESTFVNIDLEELPNLEENVHLESDDILIDEAIEDFSGAQLVFGNSRRSNTVTLPRFETVRETSLYTVDLDGFHVSYPESRAYPEVIPTEPDNTPTVIIHTEDFSGDYYLHPSLKWKKRKRAYL

SEQ ID NO: 40 - protein, 237 aa, Rabies virus
  Glycoprotein
MAPQVLPFVLLLALSMCLGKFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVADLDPYDKSLHSRVFPSGKCSGITISSTYCSTNHDYTIWMPENPRLGTSCDIFTNSRGKRASKGGKTCGFVDERGLYKS

SEQ ID NO: 41 - protein, 368 aa, Cytomegalovirus
  Glycoprotein
MMTMWCLTLFVLWMLRVVGMHVLRYGYTGIFDDTSHMTLTVVGIFDGQHFFTYHVNSSDKASSRANGTISWMANVSAAYPTYLDGERAKGDLIFNQTEQNLLELEIALGYRSQSVLTWTHECNTTENGSFVAGYEGFGWDGETLMELKDNLTLWTGPNYEISWLKQNKTYIDGKIKNISEGDTTIQRNYLKGNCTQWSVIYSGFQPPVTHPVVKGGVRNQNDNRAEAFCTSYGFFPGEINITFIHYGDKVPEDSEPQCNPLLPTLDGTFHQGCYVAIFCNQNYTCRVTHGNWTVEIPISVTSPDDSSSGEVPDHPTANKRYNTMTISSVLLALLLCALLFAFLHYFTTLKQYLRNLAFAWRYRKVRSS

SEQ ID NO: 42 - protein, 621 aa, Hepatitis C virus
  Envelope glycoproteins E1E2
IRLDGVNYATGNLPGCSFSIFLLALLSCLTIPASAYEVRNVSGVYHVTNDCSNSSIVYEAADMIMHTPGCVPCVRENNFSRCWVALTPTLAARNASVPTTTIRRHVDLLVGAAAFCSAMYVGDLCGSVFLVSQLFTFSPRQHETVQDCNCSIYPGHVSGHRMAWDMMMNWSPTAAIVVSQLLRIPQAVVDMVAGAHWGVLAGLAYYTMIGNWAKALIVMLLFAGVDGHTHVTGGSASRATRGLTALFDFGASQNLQLIKTNGSWHINRTSLNCNDSLQTGFIAALFYTHRFNSSGCPERMASCRPIDWFAQGWGPITHDKGGSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGVGNNTLICPTDCFRKHPEATYTKCGSGPWLTPRCIVDYPYRLWHYPCTVNFTIFKVRMYVGGVEHRLNAACNWTRGERCNLEDRDRSELSPLLLSTTEWQVLPCSFTTLPALSTGLIHLHQNIVDVQYLYGIGSVVVSFVIKWEYVVLLFLLLADARVCACLWMMLLIAQAEAALENLVVLNAASVAGAHSILSFLVFFCEAEF

SEQ ID NO: 43 - protein, 492 aa, Respiratory syncytial virus
  Fusion (F) protein
TEEFYQTTCSAVSKGYLSALRTGWYTSVITIELSNIKENKCNGTDAKVKLIKQELDKYKSAVTELQLLMQSTPATNNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 44 - protein, 334 aa, Zaire ebolavirus
  Spike glycoprotein
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSS

SEQ ID NO: 45 - protein, 168 aa, Zika virus
  prM protein
AEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALVAVAIAWLLGSSTSQKVIYLVMILLIAPAYS

SEQ ID NO: 46 - protein, 617 aa, Zika virus
  Serine protease NS3
SGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFEPSMLKKKQLTVLDLHPGAGKTRRVLPEIVREAIKKRLRTVILAPTRVVAAEMEEALRGLPVRYMTTAVNVTHSGTEIVDLMCHATFTSRLLQPIRVPNYNLNIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKNQEWDFVITTDISEMGANFKADRVIDSRRCLKPVILDGERVILAGPMPVTHASAAQRRGRIGRNPNKPGDEYMYGGGCAETDEGHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTKYGEKRVLKPRWMDARVCSDHAALKSFKEFAAGKR

SEQ ID NO: 47 - protein, 130 aa, Zika virus
  Serine protease subunit NS2B
SWPPSEVLTAVGLICALAGGFAKADIEMAGPMAAVGLLIVSYVVSGKSVDMYIERAGDITWEKDAEVTGNSPRLDVALDESGDFSLVEEDGPPMREIILKVVLMAICGMNPIAIPFAAGAWYVYVKTGKR

SEQ ID NO: 48 - protein, 500 aa, Zika virus
  Envelope protein E
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 49 - protein, 104 aa, Zika virus
  Capsid protein C
MKNPKEEIRRIRIVNMLKRGVARVNPLGGLKRLPAGLLLGHGPIRMVLAILAFLRFTAIKPSLGLINRWGSVGKKEAMEIIKKFKKDLAAMLRIINARKERKRR

SEQ ID NO: 50 - RNA, 669 nt, SARS coronavirus
  mRNA encoding SARS-CoV-2 Spike (S) RBD protein
agaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuuc

(19) United States
(12) Patent Application Publication
(10) Pub. No.: US 2025/0281599 A1
(43) Pub. Date: Sep. 11, 2025

(54) COMPOSITION COMPRISING ENGINEERED PLANT-DERIVED EXTRACELLULAR VESICLES AND USE THEREOF AS A VACCINE

(71) Applicant: EVOBIOTECH S.R.L., Torino (IT)

(72) Inventors: Giovanni CAMUSSI, Torino (IT); Chiara GAI, Fossano (Cuneo) (IT); Margherita Alba Carlotta POMATTO, Piossasco (Torino) (IT); Francesco Giuseppe DE ROSA, Torino (IT)

(21) Appl. No.: 18/261,572

(22) PCT Filed: Jan. 13, 2022
(86) PCT No.: PCT/EP2022/050590

§ 371 Date: Jul. 14, 2023

(30) Foreign Application Priority Data
Jan. 14, 2021 (IT) 102021000000569

(51) Int. Cl. A61K 39/215; A61K 9/1272; A61K 9/51; A61K 39/00; A61P 31/14; C12N 7/00

(52) U.S. Cl. CPC A61K 39/215; A61K 9/1272; A61K 9/5176; A61P 31/14; C12N 7/00; A61K 2039/53; A61K 2039/545; A61K 2039/55555; A61K 2039/575; C12N 2770/20034

(57) ABSTRACT

A method for treatment or prophylaxis of a disease in a subject involving administering to the subject a vaccine composition including non-immunomodulating, engineered, plant-derived extracellular vesicles (EVs), is provided. The Evs are loaded with an exogenous nucleic acid molecule encoding a protein antigen. The disease is an infectious disease or cancer. A method for the preparation of the vaccine composition, which makes use of one or more polycationic substances and one or more sugar molecules is also provided.

### CROSS-REFERENCE TO RELATED APPLICATIONS

This application is a National Stage Application of International Patent Application No. PCT/EP2022/050590, having an International Filing Date of Jan. 13, 2022, which claims priority to Italian Application No. 102021000000569 filed Jan. 14, 2021, the entire contents of which are hereby incorporated by reference herein.

### REFERENCE TO AN ELECTRONIC SEQUENCE LISTING

The contents of the electronic sequence listing named “39447_283_sequence-listing.txt”, Size: 192, 383 bytes created on Aug. 2, 2024 are herein incorporated by reference in their entirety.

### TECHNICAL FIELD

The present invention relates to compositions comprising plant-derived extracellular vesicles for use as a vaccine and/or for prophylaxis applications. More specifically, the invention relates to a composition comprising non-immunomodulating, engineered, plant-derived extracellular vesicles (EVs) loaded with an exogenous nucleic acid molecule.

### BACKGROUND

Vaccination is one of the most effective public health interventions to prevent and control infectious and non-infectious diseases. Different types of vaccines exist including live-attenuated vaccines, inactivated vaccines, subunit or recombinant or conjugate vaccines, toxoid vaccines, and those based on nucleic acids.

Nucleic acid-based vaccines comprise viral vectors, plasmid DNA, and mRNA. They have emerged as promising alternatives to conventional vaccine approaches because of their ability to induce broadly protective immune responses and their potential of being produced by rapid and flexible manufacturing processes. Among nucleic acid-based vaccine, RNA vaccines have several attributes that provide potential advantages over other vaccine types. In fact, RNA vaccines are characterized by the absence of eukaryotic contaminants. In contrast to DNA vaccines, RNA vaccines do not need to reach the nucleus to work and they are safer because plasmid DNA vaccines can integrate into the genome of the immunized host. In this context, mRNA molecules induce immune response against an encoded antigen.

The mechanism has been demonstrated for a variety of target genes including reporter genes, viral antigens, tumor antigens, and allergens. However, mRNA instability, high innate immunogenicity and inefficient in vivo delivery limit the clinical application of RNA vaccines. The main challenge faced by these vaccines is the intracellular delivery. Because of its sensitivity toward degradative enzymes, mRNA is highly unstable under physiological conditions in the body.

To date, in clinical applications mRNA molecules are vehicled by complexation with cationic polymers or using synthetic lipid nanoparticles (LNP) also called liposomes, cationic nanoparticles, EV-mimetic nanovesicles or polypeptide-based vesicles, to improve their efficacy. LNP allow mRNA protection from enzymes, promoting a higher stability, increasing RNA circulation lifetime and in vivo delivery. LNP particles are created by mixing mRNA molecules with different synthetic lipids or polymers. Nevertheless, LNP represent an inefficient delivery system.

In fact, LNP may accumulate in unintended tissues thereby limiting their effect on the target tissue of interest and LNP are characterized by a rapid clearance by the reticuloendothelial system or the mononuclear phagocyte system (Koppers-Lalic D., et al. “Virus-modified exosomes for targeted RNA delivery; a new approach in nanomedicine”. Adv Drug Deliv Rev. 2013 March;65(3):348-56). In addition, LNP can induce a pro-inflammatory response and apoptosis in vivo.

Moreover, the cellular uptake of LNP is mediated by endocytosis, which could activate the cells' autophagic-lysosomal pathway. Accumulating evidence indicates that endocytosis of nanoparticles generates autophagosomes, and their subsequent fusion with lysosomes leads to the digestion of their content.

In recent years, attempts have been made to overcome limitations of LNP by using extracellular vesicles (EVs). In fact, EVs are naturally secreted by cells and are safer compared to synthetic nanomaterials, such as LNP. EVs can exploit their natural mechanism of action and overcome some of the limitations of assembled-particles, including immunogenicity, toxicity, administration of exogenous particles, limited cell uptake and chemical assemblage of particles. The routes of EV uptake differ from those of LNP and are not likely to elicit the autophagy-lysosomal pathway, as they release their content into the cytoplasm probably without undergoing lysosomal trapping. Additionally, because of their small size, EVs can escape from rapid phagocytosis, and steadily carry and deliver nucleic acids in circulation, passing through the vascular endothelium to the target cells.

Moreover, EVs show several advantages in comparison to LNP in terms of biocompatibility, low clearance throughout the circulation, low toxicity, low safety concerns and high specificity (Sancho-Albero M, et al (2020) “Use of exosomes as vectors to carry advanced therapies”. RSC Adv 10, 23975-23987). The natural origin of EVs allows a high and inherent biocompatibility of their membrane and an efficient uptake in recipient cells. In addition, plant derived EVs can resist to stomach environment and reach the intestine after oral administration. EVs encapsulating nucleic acid molecules have been studied for multiple clinical applications, including RNA interference, RNA-based gene therapy for neurodegenerative disorders, cancer, cancer vaccine with miRNA and siRNA molecules.

US20200069594 discloses the use of plant-derived extracellular vesicles comprising a cationic polymer for delivering therapeutic agents, including coding and non-coding nucleic acid molecules.

It is well known in the art that EVs are able to protect and deliver nucleic acid molecules. For vaccine formulations, the beneficial activity of EVs is mainly based on their promoting effect on the innate and adaptive immune system cells, as shown for instance in the studies by Jesus S., et al, carried out on vaccine formulations for HBV (Jesus S., et al “Exosomes as adjuvants for the recombinant hepatitis B antigen: First report”. Eur J Pharm Biopharm. 2018 December; 133:1-11).

WO2020191361 discloses the use of EVs as vaccine to induce a cellular immune response and to treat and/or prevent a range of medical disorders.

WO2020050808 describes the use of plant-derived exosomes as adjuvants in vaccine applications along with the immunomodulating properties of these vesicles in activating or suppressing the immune system cells.

Extracellular vesicles isolated from various plants have been shown to exert a modulating activity on the cells of the immune system by reducing inflammation in the intestine. Plant sources include curcumin (Ohno M., et al, “Nanoparticle curcumin ameliorates experimental colitis via modulation of gut microbiota and induction of regulatory T cells” PLOS One. (2017) October 6;12 (10): e0185999), ginger (Zhang M., et al, (2016) “Edible ginger-derived nanoparticles: A novel therapeutic approach for the prevention and treatment of inflammatory bowel disease and colitis-associated cancer”, Biomaterials 101:321-40), orange (Berger E., et al, “Use of Nanovesicles from Orange Juice to Reverse Diet-Induced Gut Modifications in Diet-Induced Obese Mice” (2020) Mol Ther Methods Clin Dev. 18:880-892), grapes, grapefruit, and carrots (Ju S, et al. “Grape exosome-like nanoparticles induce intestinal stem cells and protect mice from DSS-induced colitis” (2013) Mol Ther. 21(7):1345-57). Moreover, EV from blueberry have shown to reduce gene expression of pro-inflammatory genes in endothelial cells stimulated with TNF-α and to protect endothelial cells from TNF-induced cytotoxicity and oxidative stress (De Robertis M, et al. “Blueberry-Derived Exosome-Like Nanoparticles Counter the Response to TNF-α-Induced Change on Gene Expression in EA.hy926 Cells” (2020) Biomolecules 10(5):742).

Notwithstanding the beneficial effects of EVs, the immunomodulating properties of these vesicles may represent a significant limitation in vaccine applications. In fact, the non-specific activation or inhibition of the immune system can be detrimental for vaccines. The use of EVs having immunosuppressive activity can significantly reduce vaccine efficiency by inhibiting immune cells response. Moreover, the development of an immune response to EVs may lead to accelerated vaccine clearance. On the contrary, immune system promotion by immune-stimulatory EVs can be harmful and led to detrimental activation and/or overreaction of a subjects' immune system. The immune responses to the vesicles can limit repeated application of vaccines. In addition, the innate immune sensing of vesicles can be associated with the inhibition of antigen expression and may negatively affect the immune response (Pardi N, et al “mRNA vaccines—a new era in vaccinology” (2018) Nat Rev Drug Discov. 17(4):261-279). Taken together, the above-illustrated evidences highlight significant disadvantages in using EVs in vaccine formulations based on the effects exhibited by these vesicles on the immune system.

### SUMMARY OF THE INVENTION

In order to overcome the limitations and draw-backs of the prior art, the present invention provides a composition comprising non-immunomodulating, engineered, plant-derived extracellular vesicles (EVs), for use as a vaccine as well as a method for the preparation of said composition as defined in the appended independent claims. The dependent claims identify further advantageous features of the claimed composition and method. The subject-matter of the appended claims forms an integral part of the present description.

### DETAILED DESCRIPTION OF THE INVENTION

The present invention relates to a composition comprising non-immunomodulating, engineered, plant-derived extracellular vesicles (EVs), wherein said extracellular vesicles (EVs) are delimited by a lipid bilayer membrane comprising an outer lipid layer and an inner lipid layer,
wherein said EVs are internally loaded with an exogenous nucleic acid molecule encoding at least one protein antigen;
wherein said EVs have a diameter ranging from 20 to 500 nm, preferably ranging from 200 to 300 nm;
wherein the membrane potential across the lipid bilayer membrane of said EVs ranges from +5 to −5 mV;
wherein ≤44% of the EVs in the composition comprise phosphatidylserine in the outer layer of the lipid bilayer membrane, for use as a vaccine.

As used herein, the term “extracellular vesicles (EVs)” refers to a heterogeneous population of particles released by virtually all living cells, which are delimited or encapsulated by a phospholipid bilayer and which carry lipids, proteins, nucleic acids and other molecules derived from the cell they are derived from. These vesicles mainly include microvesicles, released through the budding of the plasma membrane, and exosomes, derived from the endosomal compartment. Extracellular vesicles are referred to as “particles”, “microparticles”, “nanovesicles”, “microvesicles” and “exosomes”. The inherent cellular targeting properties of EVs that are dictated by their lipid composition and protein content as well as their intrinsic stability in circulation qualify these vesicles as vehicle for therapeutic agent delivery.

Within the present description the term “immunomodulation” refers to a process in which a function of the immune system is altered by enhancing (immunostimulation) or decreasing (immunosuppression) an immune response. Accordingly, the expression “non-immunomodulating EVs” as used herein refers to extracellular vesicles which do not exert any promoting nor immunosuppressive effect on the immune system.

As used herein, the term “engineered EVs” refers to extracellular vesicles which have been modified in vitro to express a heterologous component by loading a nucleic acid molecule exogenous to the vesicles' donor cells. It is therefore to be intended that an engineered EV is a non-naturally occurring vesicle.

The expression “internally loading” in the context of the present description means introducing a nucleic acid molecule in an extracellular vesicle, for example a plant-derived EV, by means, for example, of transfection, transformation or transduction.

The term “exogenous nucleic acid molecule” as used in the present description relates to a heterologous nucleic acid molecule which is not part of the natural cargo of the EVs of the invention as such. The expression “heterologous” refers to a nucleic acid molecule derived from an animal or another vegetal species than the extracellular vesicles according to the invention, or from different donor cells, different conditions, or from genetically modified donor cells.

The term “antigen protein” as used herein refers to a protein molecule capable of evoking an immune response.

In accordance with the present invention, the exogenous nucleic acid molecule loaded in the plant-derived EVs is preferably selected form the group consisting of: DNA, cDNA, messenger RNA (mRNA), pre-mRNA, long-chain RNA, coding RNA, single-stranded RNA, double stranded RNA, linear RNA, RNA oligonucleotide, self-replicating RNA (replicon RNA), retroviral RNA, a viral RNA (vRNA).

In a preferred embodiment of the invention, the exogenous nucleic acid molecule is a messenger RNA (mRNA) molecule. Within the context of the present invention, the exogenous mRNA molecule may comprise one or more modifications such as, for example, 5′ cap structure, 5′ UTR, open reading frame, 3′ UTR and poly A tail.

According to the invention, the EVs in the composition may be loaded with a single nucleic acid molecule or with a combination of two or more nucleic acid molecules.

In one preferred embodiment of the invention, the content of the loaded exogenous nucleic acid molecules in the EVs is in the range of from 20 to 200 ng/109 EVs, preferably from 30 to 100 ng/109 EVs, more preferably from 40to 60 ng/109 EVs.

The loading of exogenous nucleic acid molecules into the EVs according to the present invention may be accomplished by a number of different techniques known in the art, including, for example, electroporation, sonication, lipofectamine mediation, microinjection, co-incubation, dialysis and freeze-thaw cycles.

The present invention makes use of extracellular vesicles which have a diameter in the range of from 20 to 500 nm, preferably from 100 to 400 nm, more preferably in the range of from 200 to 300 nm.

According to the invention, the value of the membrane potential across the lipid bilayer membrane of the EVs in the composition ranges from +5 to-5 mV, preferably from +2 to −4 mV, more preferably from 0 to −3.

In a further more preferred embodiment of the invention, the value of the membrane potential of the EVs is −2 mV.

In the composition according to the invention, an amount of EVs less than or equal to (≤) 44% of the total EVs in the composition comprise phosphatidylserine in the outer layer of the lipid bilayer membrane.

Preferably, the amount of EVs in the composition comprising phosphatidylserine in the outer layer of the lipid bilayer membrane is comprised within the range of from 25% to 44% of total EVs, more preferably from 35% to 44% of total EVs, even more preferably from 40% to 44% of total EVs.

The plant-derived EVs that are used in the present invention are preferably derived from one or more plants selected from the group consisting of: genus Citrus, including lemon and orange; genus Actinidia, including kiwifruit; genus Cucurbita, including courgette; genus Brassica, including cabbage and kale; genus Punica, including pomegranate; genus Vaccinium, including blueberry, and genus Apium, including celery.

The scope of the invention includes both compositions containing EVs derived from a single plant species and compositions containing EVs derived from a plurality of plant species. It is understood that plant-derived EVs can be used in their native form or with chemical modifications.

Preferably, the plant-derived EVs in the composition according to the invention are purified from fruit juice, part of plant or culture medium of plant cells. Plant cells and parts may be derived from leaf, fruit pulp, shoot or sprout.

Suitable purification techniques of EVs include, but are not limited to, ultracentrifugation, filtration and tangential flow filtration. The selection of the most suitable method to be used for the purification of plant-derived EVs falls within the knowledge and skills of the ordinary person of skill in the art.

In one embodiment of the invention, the total protein content of the EVs in the composition of the invention is in the range of from 100 to 200 ng/10¹⁰ EVs, more preferably from 120 to 160 ng/10¹⁰ EVs.

In another embodiment, the total RNA content of the EVs in the composition of the invention is in the range of from 20 to 200 ng/10⁹ EVs, more preferably from 30 to 100 ng/10⁹ EVs, even more preferably from 40 to 60 ng/10⁹ EVs.

The expression “total protein content” encompasses both the endogenous protein cargo (internal and the membrane content of the EVs) and the loaded proteins in the EVs used in the present invention.

Within the context of the present description, the expression “total RNA content” encompasses both the endogenous RNA cargo and the loaded exogenous RNA in the EVs according to the invention.

As further illustrated in the Experimental Section below, the present inventors have surprisingly found that the engineered, plant-derived EVs having the structural and functional features as above defined do not exhibit any immunomodulatory activity, i.e. they are devoid of any ability to affect the cells of the immune system neither promoting nor reducing the activation and efficacy of these cells.

Differently from native plant-derived EVs, the non-naturally occurring EVs according to the invention are advantageously capable to deliver antigenic molecules to target cells without exerting per se any effect on the cells of the immune system. Therefore, the use of the EVs according to the invention enables to overcome the safety concerns in connection with EV-based vaccine formulations and to avoid detrimental activation or inhibition of the immune system, thereby enhancing the efficacy of vaccines.

Moreover, the EVs according to the invention are proved to efficiently load and vehicle nucleic acid agents to recipient cells and protect them from environment degradation. In particular, the high resistance to stomach environment allows the oral administration of the composition according to the invention.

After administration, the interaction of the loaded EVs with antigen presenting cells (APC), including macrophages and dendritic cells, allows the transfer of the nucleic acid molecules to the antigen-presenting cell. In the target cells, the nucleic acid molecules, comprising DNA and mRNA molecules, are expressed leading to protein antigen translation. Then, the antigen is presented on the surface of the APC inducing the specific activation of immune cells direct against the tumor cells or pathogen allowing an efficient immune protection.

Thanks to the advantageous features of the non-naturally occurring EVs as above illustrated, the composition of the invention is particularly suitable for use as a vaccine.

In the context of the present invention, the composition of the invention may be used as a vaccine for the treatment of an existing disease or prophylactically to prevent the occurrence of this disease.

Exemplary protein antigens encoded by the exogenous nucleic acid molecules encapsulated into the EVs of the invention include, but are not limited to, bacterial, viral, fungal, protozoan and tumor antigens, mammalian homologs thereof, and homologs from animals of veterinary or industrial interest thereof.

Accordingly, the composition of the present invention is particularly useful for the treatment or prophylaxis of infectious diseases or cancer diseases.

Exemplary cancer diseases include, but are not limited to, bladder cancer, cervical cancer, renal cell cancer, testicular cancer, colorectal cancer, lung cancer, head and neck cancer, ovarian, lymphoma, liver cancer, glioblastoma, melanoma, myeloma, leukemia, pancreatic cancer.

By way of example, but without limitation, the infectious disease may be, a viral disease, a bacterial disease, a fungal disease or a protozoan disease, such as, for example, COVID-19 disease, influenza, HPV infection, HIV infection, rhinovirus infection, hepatitis, flavivirus infections, encephalitis, meningitis, gastroenteritis, cholera, diphtheria, chlamydia, tuberculosis, typhoid, Sexually Transmitted Infections (STI), malaria, mycoses, toxoplasmosis.

In one embodiment of the invention, the at least one antigen encoded by the exogenous nucleic acid molecule loaded into the EVs is a tumor antigen selected from the group consisting of human kallikrein related peptidase 3, also called prostate specific antigen (PSA), human prostate stem cell antigen (PSCA), human prostate specific membrane antigen (PSMA), human metalloreductase (six transmembrane epithelial antigen of the prostate 1 (STEAP1), human Receptor tyrosine-protein kinase erbB-2, also called Tyrosine kinase-type cell surface receptor HER2, human cell surface associated mucin 1 protein (MUC1), also called Breast carcinoma-associated antigen DF3, human Tyrosinase-related protein 2 (TRP-2), human Serine/threonine-protein kinase B-raf, also called Proto-oncogene B-Raf, human Mast/stem cell growth factor receptor Kit, also called Proto-oncogene c-Kit, human GTPase NRas, also called Transforming protein N-Ras, human melanoma-associated antigen 1, human melanoma-associated antigen 1 protein, human NY-ESO-1 protein, and any combination thereof.

In another embodiment of the invention, the at least one protein antigen is a bacterial antigen from a bacterium selected from the group consisting of Staphylococcus aureus, Mycobacterium tuberculosis, Chlamydia trachomatis, Streptococcus pyogenes, Streptococcus pneumoniae, Borrelia burgdorferi, Borrelia mayonii (e.g., Lyme disease), Klebsiella sp., Pseudomonas aeruginosa, Enterococcus sp., Proteus sp. (e.g. vulgaris, mirabilis, penneri), Neisseria gonorrhoeae, Enterobacter sp., Actinobacter sp., coagulase-negative Staphylococci (CoNS), Mycoplasma sp., Clostridium difficile, Bacillus anthracis, Vibrio cholerae, Clostridium botulinum, Clostridium tetani, Salmonella sp., Treponema pallidum, Plasmodium sp., and any combination thereof.

In yet another embodiment of the invention, the at least one protein antigen is a fungal antigen from a fungus selected from the group consisting of Blastomyces, Cryptococcus gattii, Cryptococcus neoformans, Fusarium, Aspergillus, Candida, Candida albicans, Candida auri, Cryptococcus, Histoplasma, Blastomyces, Coccidioides, Mucormycetes, Pneumocystis jirovecii, dermatophyte, Sporothrix, and any combination thereof.

In a still another embodiment, the at least one protein antigen is a protozoan antigen from a protozoa selected from the group consisting of Plasmodia species (e.g., vivax and falciparum), Giardia intestinalis, Hexamita salmonis, Histomonas meleagridis, Trichomonas foetus, Dientamoeba fragilis, Trichomonas vaginalis, Leishmania, Trypanosoma cruzi, Trypanosoma brucei rhodensiense, Trypanosoma brucei gambiense, Plasmodium parasite, Entamoeba histolytica, Naeglaria, Acanthomoeba, Peronosporomycetes, Phytophthora infestans, Giardia lamblia, Giardia duodenalis, Toxoplasma gondii, Balantidium Coli, Theileria parva, Theileria annulate, Phipicephalus appendiculatus, Prototheca moriformis, and any combination thereof.

Preferably, the protozoan antigen is selected from the group consisting of dense granule protein 6 (GRA6), rhoptry protein 2A (ROP2A), rhoptry protein 18 (ROP18), surface antigen 1 (SAG1), surface antigen 2A (SAG2A), apical membrane antigen 1 (AMA1) of Toxoplasma gondii, and any combination thereof.

In a still further embodiment, the at least one protein antigen is a viral antigen from a virus selected from the group consisting of Human Papilloma Virus (HPV), Human Immunodeficiency virus HIV (e.g. HIV-1, HIV-2), Hepatitis A virus, Hepatitis B virus (HBV), Hepatitis C Virus, Hepatitis D Virus, Hepatitis E Virus, Herpes virus (Human Gamma herpes virus 4 (Epstein Barr virus), herpes simplex virus 2 (HSV2), human herpes virus 8, Influenza Virus (e.g. influenza A virus, influenza B virus), cytomegalovirus, Crimean-Congo hemorrhagic fever orthonairovirus, corona viruses, Human polyomavirus 2, BK virus, Severe acute respiratory syndrome coronavirus (SARS-CoV, SARS-Cov-2, COVID-19), Middle East respiratory syndrome-related coronavirus (MERS-CoV), noroviruses, filoviruses (Cueva, Marburg and Ebola virus), Chikungunya virus, Human alphaherpesvirus 3 (HHV-3) or varicella-zoster virus (VZV), Rubella virus, Merkel cell polyomavirus (MCV), bunyavirus (e.g., hanta virus), arena virus (e.g., lymphocytic choriomeningitis mammarenavirus (LCMV) and Lassa virus), flavivirus (Dengue virus, Zika virus, Japanese encephalitis, West Nile, Tick-borne encephalitis virus (TBEV) and Yellow fever), rhinovirus, Human parainfluenza viruses (HPIVs), enterovirus (e.g polio), Respiratory syncytial virus (RSV), Mumps virus, Coxsackievirus, Measles virus, astrovirus (e.g., gastroenteritis), rhabdoviridae (e.g., rabies), Adenovirus, Adeno-associated virus (AAV), oncogenic viruses, including human papillomavirus, hepatitis B virus, hepatitis C virus, Epstein-Barr virus, Kaposi's sarcoma-associated herpesvirus, human T-lymphotropic virus and Merkel cell polyomavirus, and any combination thereof.

According to a preferred embodiment of the invention, the viral antigen is selected from the group consisting of Spike proteins also called Surface Glycoprotein of Severe acute respiratory syndrome coronavirus 2 or SARS-COV-2 or COVID-19, N protein also called Nucleocapside phosphoprotein of Severe acute respiratory syndrome coronavirus 2 or SARS-COV-2 or COVID-19, M protein also called Membrane Glycoprotein of Severe acute respiratory syndrome coronavirus 2 or SARS-COV-2 or COVID-19, Hemagglutinin (HA) protein of influenza A virus H5N1, Hemagglutinin (HA) protein of influenza A virus H3N2, Hemagglutinin (HA) protein of influenza A virus H1N1, Hemagglutinin (HA) protein of influenza A virus H7N9, Hemagglutinin (HA) protein of influenza A virus H1N1, Hemagglutinin (HA) protein of influenza A virus H2N2, Hemagglutinin (HA) protein of influenza B virus, Neuraminidase (NA) protein of influenza A virus H5N1, Neuraminidase (NA) protein of influenza A virus H1N1, Neuraminidase (NA) protein of influenza A virus H3N2, Neuraminidase (NA) protein of influenza A virus H7N9, Neuraminidase (NA) protein of influenza A virus H9N2, Neuraminidase (NA) protein of influenza A virus H2N2, Neuraminidase (NA) protein of influenza A virus H1N1, Neuraminidase (NA) protein of influenza B virus, envelope protein of Human immunodeficiency virus (HIV1), envelope protein of Human immunodeficiency virus (HIV2), Major Capsid Protein L1 of Human Papilloma Virus (HPV), Minor Capsid Protein L2 of Human Papilloma Virus (HPV), glycoprotein of Rabies lyssavirus, glycoprotein of Human Cytomegalovirus, envelope glycoproteins E1E2 of Hepatitis C virus, Fusion protein (F) of Respiratory syncytial virus (RSV), spike glycoprotein of Zaire ebolavirus, Protein prM of Zika virus, Serine protease NS3 of Zika virus, Serine protease subunit NS2B of Zika virus, Envelope protein E of Zika virus, Capsid protein C of Zika virus, SARS-CoV-2 Spike(S) RBD protein, and any combination thereof.

In an exemplary embodiment, the encoded at least one protein antigen as above defined comprises, consists essentially or consists of an amino acid sequence selected from the group consisting of SEQ ID NOs.: 1-13, 15, 16, 18, and 20-49.

In another embodiment of the invention, the exogenous nucleic acid molecule loaded in the EVs is a mRNA molecule comprising or consisting of a nucleotide sequence selected from the group consisting of SEQ ID NOs. 14, 17, 19 and 50. More particularly, SEQ ID NOs. 14, 17, 19 and 50 correspond to mRNA sequences coding for SARS-COV-2 S protein, N protein, M protein and Spike(S) RBD protein, respectively.

According to the present invention, it is envisaged that the composition may comprise engineered, plant-derived EVs loaded with a single exogenous nucleic acid molecule or, alternatively, a combination of engineered, plant-derived EVs loaded with different exogenous nucleic acid molecules.

It is understood that the protein antigen within the scope of the invention may comprise one or more modifications in order to improve antigen immunogenicity and/or stability. Exemplary modifications include post-translational modifications.

The composition according to the invention may be used alone or in combination with other vaccines.

In one embodiment, the composition according to the invention further comprises one or more polycationic substances, said one or more polycationic substances being associated with the outer lipid layer of the lipid bilayer membrane of the EVs through electrostatic interactions.

Preferably, the one or more polycationic substances are selected from the group consisting of cationic proteins, including protamine, calcitonin peptides, plectasin, lactoferrin, protamine-like proteins, such as spermine or spermidine, nucleoline, histones, cell penetrating peptides (CPPs); cationic peptides, including histidine-rich peptides, arginine-rich peptides, lysine-rich peptides, cationic arginine-rich peptides (CARPs); polypeptides, including poly-arginine, poly-lysine, poly-histidine, histidine-rich peptides, arginine-rich peptides, lysine-rich peptides; polysaccharides, including chitosan, glycosaminoglycan such as polysulfated glycosaminoglycan (PSGAG), cationic dextrans; glycerol, polyethylene glycol (PEG).

A preferred polycationic substance is protamine.

Preferably, the content of the one or more polycationic substances in the composition is in the range of from 0.001 to 2 μg/10¹⁰ EVs, more preferably from 0.05 to 1 μg/10¹⁰ EVs, even more preferably from 0.1 to 0.4 μg/10¹⁰ EVs.

According to the invention, the one or more polycationic substances may be used alone or in combination. It is understood that the polycationic substance can be used in its native form or with chemical modifications. Such components may be used individually or in combination.

In another embodiment according to the invention, the EVs in the composition of the invention are additionally loaded with one or more sugar molecules, said one or more sugar molecules being associated with the exogenous nucleic acid molecule loaded into the EVs through electrostatic interactions and hydrogen bonding.

Preferably, the one or more sugar molecules are selected from the group consisting of disaccharides, including trehalose, maltose, lactose, sucrose, cellobiose, chitobiose, kojibiose, nigerose, isomaltose, β,β-trehalose, α,β-trehalose, sophorose, laminaribiose, gentiobiose, trehalulose, turanose, maltulose, leucrose, iso-maltulose, gentiobiulose, mannobiose, melibiose, melibiulose, rutinose, rutinulose, xylobiose; sugar alcohols, including arabitol, erythritol, glycerol, HSHs, isomalt, lactitol, maltitol, mannitol, sorbitol, xylitol; polysaccharides, including starch, glycogen, galactogen, inulin, arabinoxylans, cellulose, chitin and pectin.

A preferred sugar molecule is trehalose. Trehalose is a non-reducing disaccharide sugar commonly used as a cytoprotectant to stabilize proteins and nucleic acids. Additionally, trehalose can resolve secondary structures of RNA.

Preferably, the content of the one or more sugar molecules in the EVs according to the invention is in the range of from 0.1 to 10 mg/10¹⁰ EVs, more preferably from 0.5 to 5 mg/10¹⁰ EVs, even more preferably from 1 to 2 mg/10¹⁰ EVs.

In another embodiment, the content of the one or more sugar molecules in the EVs according to the invention is in the range of from 0.1 to 20 mg/μg of loaded exogenous nucleic acid, preferably from 1 to 10 mg/μg of loaded exogenous nucleic acid, more preferably from 2 to 6 mg/μg of loaded exogenous nucleic acid.

It is understood that the sugar molecules can be used in their native form or with chemical modifications. Such components may be used individually or in combination.

Before usage, the non-naturally occurring EVs in the composition of the invention may be lyophilized and resuspended with water. Alternatively, the non-naturally occurring EVs used in the composition of the invention may be freshly prepared or stored at 4° C., −20° C. or −80° C.

The composition according to the present invention may be formulated in several administrable forms, including powders, granules, tablets, capsules, suspensions, emulsions, syrups, aerosols, pills, sugar-coated tablets, capsules, liquids, gels, syrups, slurries, and suspensions.

The composition of the invention may optionally contain suitable excipients, preservatives, solvents or diluents according to conventional method.

Exemplary excipients include, but are not limited to, sugars, including sucrose, D-mannose, D-fructose, dextrose, anhydrous lactose, D-trehalose, D-sorbitol; proteins, including human serum albumin, hydrolyzed casein, MRC-5 cellular proteins, hydrolyzed gelatin, CRM197 carrier protein, proteins from plants, yeast, bacteria, eggs; essential and non-essential aminoacids such as asparagine, phenylalanine, arginine, histidine; sodium, including sodium chloride, sodium bicarbonate, sodium carbonate, sodium borate, sodium benzoate, sodium taurodeoxycholate, sodium deoxycholate, monobasic sodium phosphate, dibasic sodium phosphate, sodium metabisulphite; potassium, including potassium phosphate, polacrilin potassium, monobasic and dibasic potassium phosphate, potassium chloride; magnesium stearate, calcium chloride, calcium phosphate, calcium silicate, glutamate, cellulose, microcrystalline cellulose, cellulose acetate phthalate, aluminum, aluminum hydroxide, aluminum phosphate, amorphous aluminum hydroxyphosphate sulfate, potassium aluminum sulfate, citric acid, iron ammonium citrate, castor oil, neomycin, streptomycin, aminoglycoside, kanamycin, gentamicin, chlortetracycline, amphotericin B, plasdone C, alcohol, acetone, benzethonium chloride, formaldehyde, glycerin, ascorbic acid, trometamol, urea, glutaraldehyde, 2-phenoxyethanol, polysorbate 80 (Tween 80), polymyxin B, ammonium thiocyanate, tromethamine, host cell DNA benzonase, formalin, phosphate-buffered saline solution, polysorbate 20, deoxycholate, dibasic dodecahydrate, monobasic dehydrate, formalin, polymyxin B, beta-propiolactone, hydrocortisone, squalene, sorbitan trioleate, barium, cetyltrimethylammonium bromide (CTAB), octoxynol-10 (TRITON X-100), α-tocopheryl hydrogen succinate, cetyltrimethlyammonium bromide, and β-propiolactone, thimerosal, ethylenediaminetetraacetic acid (EDTA), phenol, beta-propiolactone, DMEM, HEPES, polydimethylsiloxane, vitamins, dioleoyl phosphatidylcholine (DOPC), 3-O-desacl-4′monophosphoryl lipid A (MPL), lipids, cholesterol, panthenol, gums, including guar gum, boric acid and borates, including sodium tetraborate, glycerol, allantoin, triethanolamine, alginate, pluronic, poloxamer, including P188, P331; PEG, including PEG8000; glycols, including ethylene glycol, propylene glycol, and glycerol; citicoline (cyti-dine-5-diphosphocholine; CDP-choline), cholesterol.

Illustrative, non-limiting examples of preservatives suitable for use in the composition of the invention include parabens, including ethyl paraben, methyl paraben, propyl paraben, formaldehyde donors including DMDM hydantoin, imidazolidinyl urea, and glutaraldehyde, phenol derivatives, benzoic acid, benzyl alcohol.

Suitable solvents or diluents to be used in the invention may be selected from purified water, ethanol and benzyl alcohol.

According to the invention, it is contemplated that an adjuvant can be added to the composition for use as a vaccine.

Illustrative, non-limiting examples of adjuvants suitable for use in the immunogenic composition of the invention are mineral compositions, including aluminum salts such as aluminium hydroxide, aluminium potassium phosphate, AS04, and others, calcium salts, hydroxides (e.g. oxyhydroxides), phosphates (e.g. hydroxyphoshpates, orthophosphates), sulphates; emulsions, including oil-in-water and water-in-oil emulsions, such as Freund's adjuvant, complete Freund's adjuvant, incomplete Freund's adjuvant, MF59, AF03, AS03, AS02, glucopyranoside lipid adjuvant (GLA-SE), glucopyranosyl lipid adjuvant (GLA);

bacterial or microbial derivatives, including non-toxic derivatives of enterobacterial lipopolysaccharide (LPS), monophosphoryl lipid A (MPL), 3-O-deacylated MPL (3dMPL), lipid A, lipid A from Escherichia coli such as OM-174. OM-174; immunostimulatory oligonucleotides, including nucleotide sequences containing a CpG motif, bacterial double stranded RNA, oligonucleotides containing palindromic or poly(dG) sequences, ADP-ribosylaling toxins and detoxified derivatives, RC529; cyclic GMP-AMP adjuvant, STING agonists, CAF01, immunostimulating complexes (ISCOMs), ISCOMATRIX, AS01; polyoxyethylene ether and polyoxyethylene ester formulations, polymeric particles, such as poly(lactide-co-glycolide) (PLG) microparticles, polyphosphazene (PCPP), saponin formulations, such as saponin derived from Smilax ornata (sarsaprilla), Gypsophilla paniculata (brides veil), and Saponaria officinalis (soap root), purified formulations, such as QS7, QS17, QS18, QS21, QH-A, QH-B and QH-C; human immunomodulators, including cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor; bioadhesives and mucoadhesives, including esterified hyaluronic acid microspheres, or mucoadhesives such as cross-linked derivatives of poly(acrylic acid), polyvinyl alcohol, polyvinyl pyrollidone, polysaccharides and carboxymethylcellulose, chitosan and derivatives thereof; t muramyl peptides, including N-acetyl-muramyl-Lthreonyl-D-isoglutamine (thr-MDP), N-acetyl-normuramyl-L-alanyl-D-isoglutamine (nor-MDP), and N-acetylmuramyl-Lalanyl-D-isoglutaminyl-L-alanine-2-(1′-2′-dipalmitoyl-sn-glycero-3-hydroxyphosphoryloxy)-ethylamine MTP-PE); imidazoquinolone compounds, including Imiquamod and its homologues; virosomes and Virus Like Particles (VLPs).

The composition of the invention may be administered via various routes, including oral, intranasal, parenteral, including subcutaneous, intraperitoneal, intravenous, intradermal, intramuscular, intrasplenic, and intranodal.

Preferably, the pharmaceutical composition of the present invention is in a form suitable for oral, intranasal or parenteral administration.

The administration dose, the number and frequency of applications are determined according to various factors, such as the disease to treat or prevent and the patient's characteristics, and can be determined by a person of ordinary skill in the art by using his/her normal knowledge.

In addition, the composition according to the invention may be lyophilized and is stable without the need of a cold-chain storage.

Within the scope of the present invention is also a method for the preparation of a composition having the features as above-defined.

According to the invention, the method comprises the steps of:
(i) contacting and mixing a suspension of plant-derived extracellular vesicles (EVs) with one or more polycationic substances to obtain a first mixture;
(ii) contacting and mixing a preparation of nucleic acid molecules with one or more sugar molecules to obtain a second mixture, said nucleic acid molecules encoding at least one protein antigen;
(iii) admixing said first mixture and said second mixture to obtain a third mixture; and
(iv) adding to said third mixture a pre-determined volume of water, wherein the ratio of said pre-determined volume of water to the volume of the third mixture is comprised within 5:1 to 15:1, preferably within 8:1 to 12:1.

A preferred ratio of the pre-determined volume of water to the volume of the third mixture is 10:1.

Optionally, the method according to the invention may further comprise concentrating the composition obtained in step (iv). Concentration techniques are well known and include, for example, filtration, ultracentrifugation, tangential flow filtration, chromatography and precipitation. The skilled person will be aware of techniques for concentrating a composition, and any such suitable method may be used.

In one embodiment of the method of the invention, in step (i) mixing further comprises the step of incubating the first mixture for a time ranging from 30 minutes to 2 hours, preferably for 1 hour, at a temperature ranging from 30 to 40° C., preferably at 37° C.

In another embodiment of the method of the invention, in step (ii) mixing further comprises the step of incubating the second mixture for a time ranging from 5 to 30 minutes, preferably for 10 minutes, at a temperature ranging from 0 to 25° C., preferably at 20° C.

In yet another embodiment of the method of the invention, in step (iii) admixing further comprises the step of incubating the third mixture for a time ranging from 1 to 5 hours, preferably 3 hours, at a temperature ranging from 30 to 40° C., preferably at 37° C.

In a further embodiment of the method of the invention, step (iv) further comprises an incubation step performed for a time ranging from 5 to 24 hours, preferably 12 hours, at a temperature ranging from 0 to 10° C., at 4° C.

Suitable polycationic substances and sugar molecules for use in the method according to the invention are as above described with reference to the composition.

Without wishing to be bound by any theory, the inventors believe that the polycationic substance may alter the charge of the lipid bilayer membrane of the plant-derived EVs and allow the adsorption of the nucleic acid molecules on the outer surface of such membrane. Further, the inventors believe that the sugar may play a protecting role of nucleic acid molecules in order to allow an efficient introduction of these molecules into the plant-derived EVs.

Preferably, the concentration of plant-derived EVs in the first mixture is comprised within the range of from 5×10¹⁰ to 10¹² EVs/ml on the total volume of said first mixture, more preferably from 1×10¹¹ to 5 ×10¹¹ EVs/ml on the total volume of said first mixture.

The first mixture according to the method of the invention may further comprise a salt, preferably NaCl, more preferably NaCl at a concentration of 0.9% (w/v) on the total volume of said first mixture.

In one embodiment of the method of the invention, the one or more polycationic substances are present in the first mixture at a concentration comprised within the range of from 0.1 to 2 μg/ml on the total volume of said first mixture, preferably from 0.1 to 1 μg/ml on the total volume of said first mixture, more preferably from 0.4 to 0.6 μg/ml on the total volume of said first mixture.

In another embodiment of the invention, the nucleic acid molecule is present in the second mixture at a concentration comprised within the range of from 0.1 to 10 μg/ml on the total volume of said second mixture, preferably from 0.1 to 1 μg/ml on the total volume of said second mixture, more preferably from 0.1 to 0.5 μg/ml on the total volume of said second mixture.

In a still another embodiment according to the invention, the one or more sugar molecules are present in the second mixture at a concentration comprised within the range of from 1 to 20% (w/v) on the total volume of said second mixture, preferably from 1 to 10% (w/v) on the total volume of said second mixture, more preferably from 1 to 5% (w/v) on the total volume of said second mixture.

According to the method of the invention, the mixing of the suspension comprising plant-derived EVs with the polycationic substance in step (i) and/or the mixing of the preparation of nucleic acid molecules with one or more sugar molecules in step (ii) may be performed by vortexing, preferably for a period of time of at least 30 seconds.

According to the invention, it is envisaged that the method may comprise further manipulations to improve the loading of nucleic acid molecules into plant-derived EVs including, but not limited to, electroporation, sonication, transfection, incubation, cell extrusion, saponin-mediated permeabilization, and freeze-thawing.

Another aspect of the present invention is a composition comprising non-immunomodulating, engineered, plant-derived extracellular vesicles (EVs), obtainable by a method as above defined, for use as a vaccine.

### EXAMPLES

The following experimental section is provided purely by way of illustration and is not intended to limit the scope of the invention as defined in the appended claims. In the following experimental section, reference is made to the appended drawings, wherein:

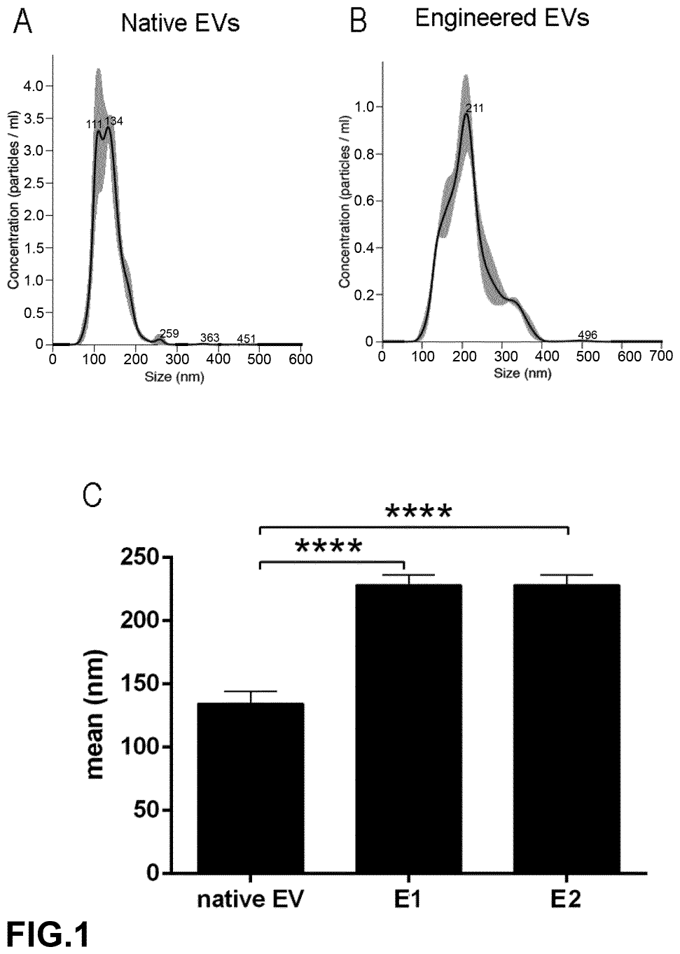

FIG. 1 shows the characterization of engineered, plant-derived EVs of the invention in experimental example 1 compared to native plant-derived EVs. Representative image of Nanosight analysis of native EVs (A) and engineered EVs of the invention (B) from kiwifruit showing a significant difference in size. Statistical analysis of the mean diameter of n=3 preparations of native plant-derived EVs and engineered, plant-derived EVs of the invention analyzed by Nanosight (C). E1=engineered EVs from cabbage EV, E2=engineered EVs from blueberry EV. p: **** <0.001.

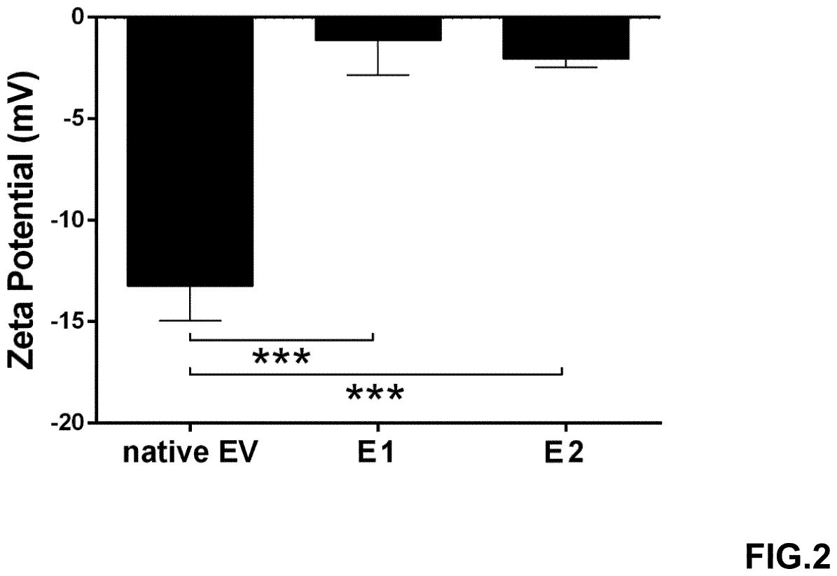

FIG. 2 shows the values of the membrane potential across the lipid bilayer membrane (Z potential) measured in EVs in experimental example 1. The membrane potential was measured as mVolt (mV) in native EVs (native EV) and engineered EVs of the invention from courgette (E1) and blueberry (E2). The statistical significance was calculated comparing the membrane potential measured for engineered plant-derived EVs with the values determined for native plant-derived EVs. p: *** <0.005. N=3 experiments were performed for each data set. Data are shown as mean±standard deviation (SD).

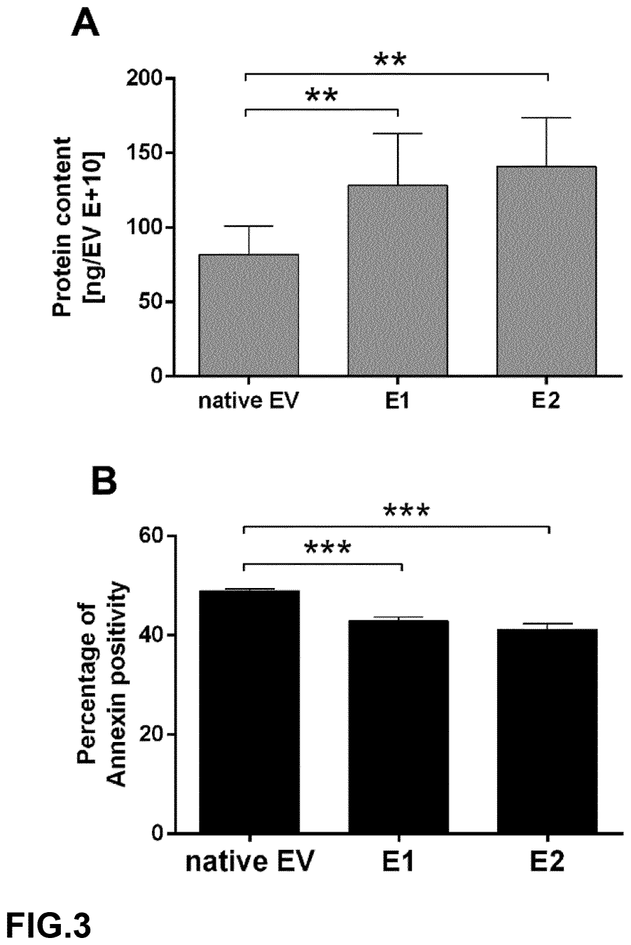

FIG. 3A shows the protein content of native plant-derived EVs and engineered plant-derived EVs of the invention in experimental example 1 expressed as nanograms (ng) of protein in 10¹⁰ EVs. Measurements were performed on native plant-derived EVs (native EV) and engineered, plant-derived EVs from pomegranate (E1) and kiwifruit (E2). The statistical significance was calculated comparing the protein content of engineered plant-derived EVs with the values measured in native plant-derived EVs. p: ** <0.01. N=3 experiments were performed for each data set. FIG. 3B shows the percentage of EVs in the composition of the invention in experimental example 1 containing phosphatidylserine in the outer layer of the lipid bilayer membrane. The presence of phosphatidylserine in the outer layer of vesicles membrane was analyzed in compositions comprising native plant-derived EVs (native EV) and compositions comprising engineered, plant-derived EVs from cabbage (E1) and blueberry (E2). In each sample, the phosphatidylserine content was measured using cytofluorimetric assay (FACS) as staining to Annexin V and expressed as percentage of fluorescent signal. The statistical significance was calculated comparing the percentage of engineered plant-derived EVs containing phosphatidylserine with native plant-derived EVs. p: ** <0.01. N=3 experiments were performed for each data set. Data are shown as mean±standard deviation (SD).

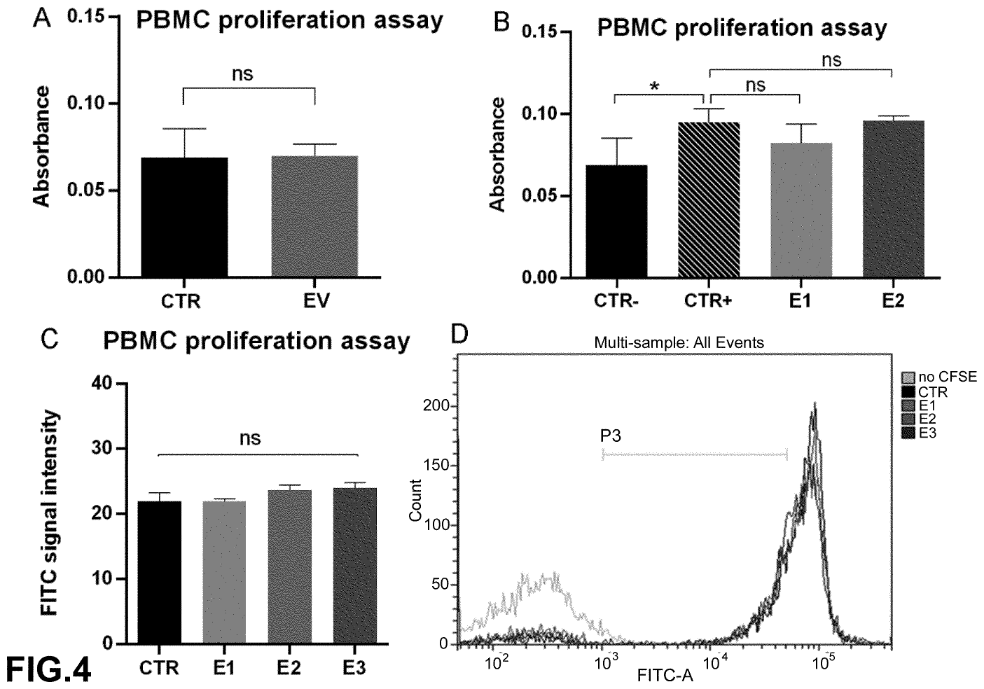

FIG. 4 shows the results of the immunomodulatory assay on engineered plant-derived EVs of the invention in experimental example 1. PBMC cells were incubated with engineered plant-derived EVs from pomegranate (dose of 50,000 particles/cell) for 48 hours and cellular proliferation was measured by BrdU incorporation. (A) The histogram shows the absorbance (mean±SD) for untreated PBMC (CTR) and PBMC stimulated with the EVs of the invention. Absorbance is directly proportional to cell proliferation. The proliferation rate of PBMC stimulated with the EVs of the invention is unchanged and not statistically significant compared to control (CTR). Then, lymphocytes were activated with LPS (dose of 100 ng/ml) and stimulated with the EVs of the invention (dose of 50,000 particles/cell) for 48 hours and proliferation was measured by BrdU incorporation. (B) The histogram shows the absorbance (mean±SD) of non-stimulated PBMC (CTR−), PBMC treated with LPS (CTR+), PBMC treated with LPS and the EVs of the invention from pomegranate (E1), and PBMC treated with LPS and the EVs of the invention from kiwifruit (E2). Absorbance is directly proportional to cell proliferation. LPS significantly activates PBMC proliferation compared to untreated cells, while the proliferation rate of PBMC stimulated with LPS and the EVs of the invention is unchanged and not statistically significant compared to PBMC treated with LPS. PBMC proliferation was also measured by using the fluorescent dye CFSE. PBMC were stimulated with the EVs of the invention (dose of 50,000 vesicles/cell) for 24 hours, then proliferation was analyzed by flow cytometry (C, D). The histogram (C) shows fluorescent FITC intensity (mean±SD) for untreated PBMC (CTR) and PBMC stimulated with the EVs of the invention from cabbage (E1), celery (E2), and courgettes (E3). The proliferation rate of PBMC stimulated with the different samples of EVs of the invention is unchanged and not statistically significant compared to CTR. In fact, the histograms of flow cytometry analysis of CTR, E1, E2, and E3 were completely overlapping (D). p: * <0.05; ns>0.05.

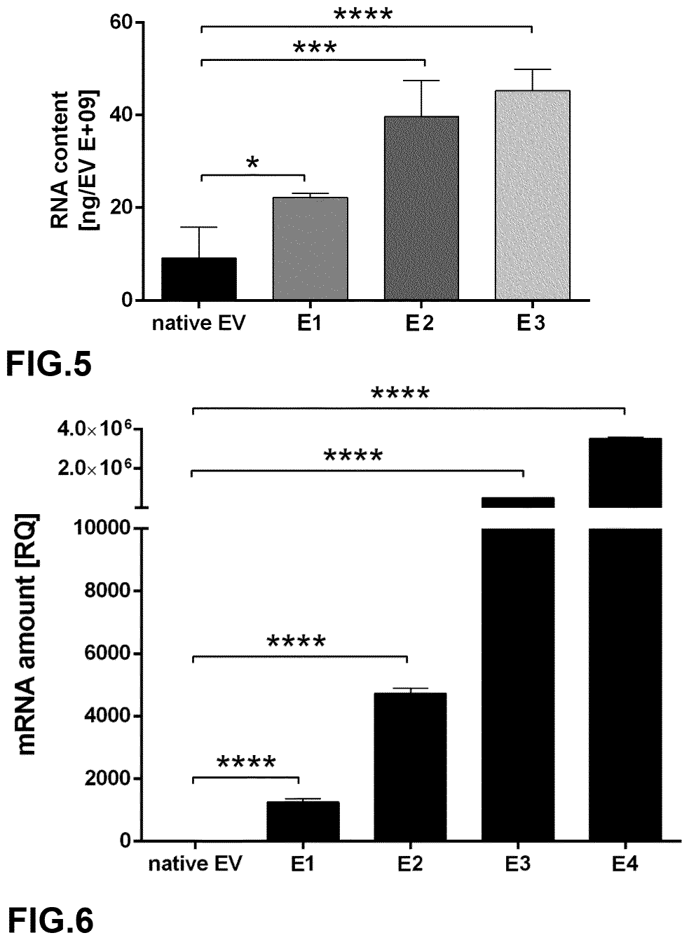

FIG. 5 shows the total RNA content of EVs in experimental example 1. The total RNA content was measured in native plant-derived EVs and engineered plant-derived EVs from celery (E1), pomegranate (E2) and kiwifruit (E3). The total RNA content was measured with absolute quantification of the RNA contained in each sample after the RNA extraction and was expressed as the amount of RNA (ng) normalized for the number of vesicles (ng/10⁹ vesicles). The statistical significance was calculated comparing the total RNA content value of each sample of EVs of the invention with native plant-derived EVs. p: * <0.05, *** <0.005, **** <0.001. N=3 experiments were performed for each data set. Data are shown as mean±standard deviation (SD).

FIG. 6 shows the quantification of exogenous nucleic acid molecules loaded in the engineered, plant-derived EVs of the invention in experimental example 2. For the assay, mRNA molecules coding for the nucleocapsid (N) protein of SARS-CoV-2 were used and loaded nucleic acid molecules were measured by qRT-PCR in native plant-derived EVs (native EV) and engineered, plant-derived EVs from kiwifruit (E1 and E3) and celery (E2 and E4). Two different doses of mRNA were used: 0.1 μg/ml for samples E1 and E2, and 1 μg/ml for samples E3 and E4. The amount of loaded mRNA was expressed as RQ value accordingly to method described. The statistical significance was calculated comparing the amount of loaded nucleic acid in each sample of EVs of the invention with native plant-derived EVs. p: **** <0.001. N=3 experiments were performed for each data set. Data are shown as mean±standard deviation (SD).

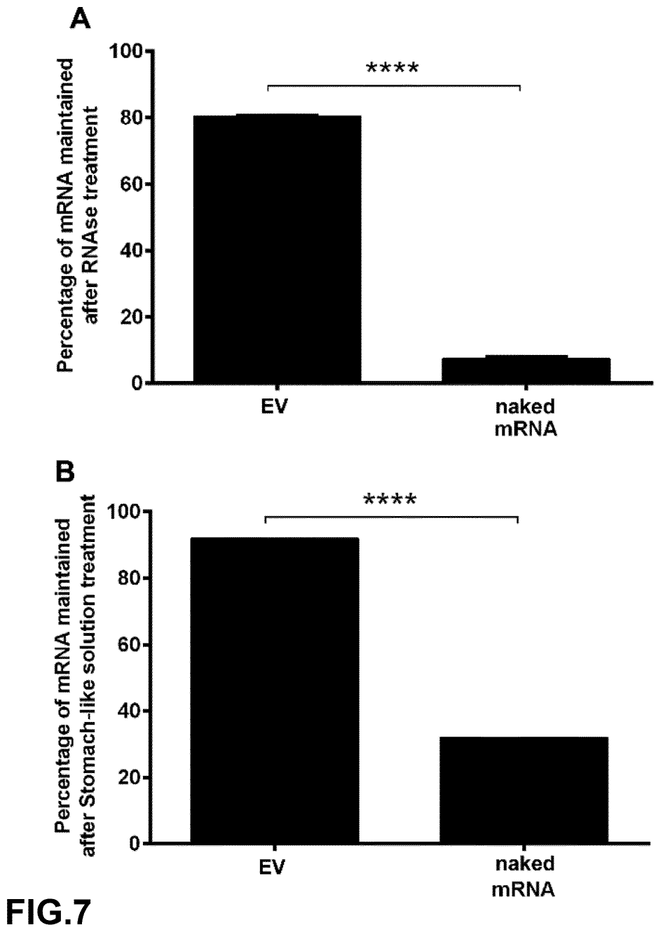

FIG. 7 shows the resistance of the nucleic acid molecules loaded in engineered, plant-derived EVs of the invention to degrading environments in experimental example 3. For the experiments, mRNA molecules were used and measured by qRT-PCR assay. Graphs indicate the percentage of mRNA molecules still present after the degrading assay in comparison to the starting material and show that a total of 100% of mRNA is preserved in the EVs of the invention. (A) The resistance to enzyme degradation was measured after treatment with RNAse, whereas (B) the resistance to gastrointestinal environment was evaluated after the treatment with a stomach-like solution. In all experiments, naked mRNA was used as control. The experiments were performed on engineered, plant-derived EVs from pomegranate (7A) and kiwifruit (7B). The statistical significance was calculated comparing the percentage of the nucleic acid preserved in the EVs of the invention with naked mRNA. p: **** <0.001. N=3 experiments were performed for each data set. Data are shown as mean±standard deviation (SD).

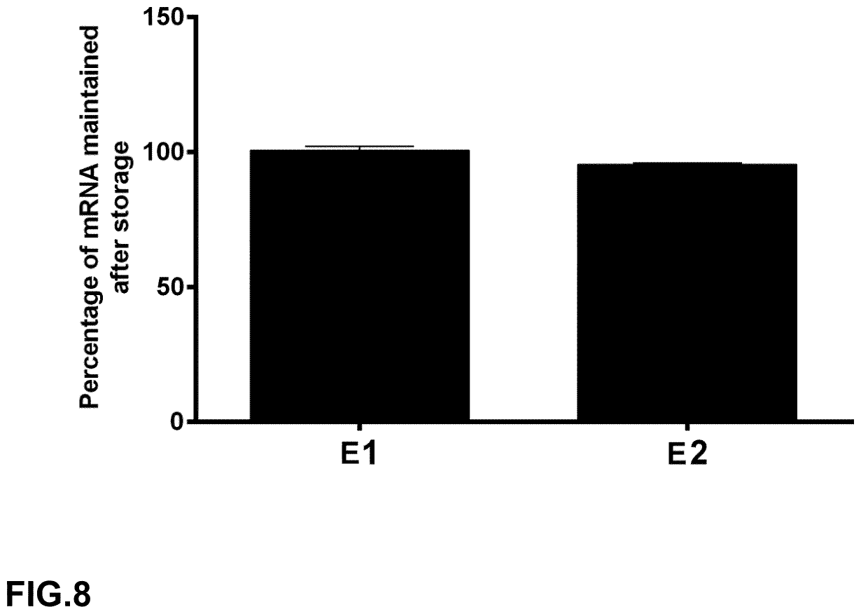

FIG. 8 shows the resistance of nucleic acid molecules loaded in engineered, plant-derived EVs of the invention to storage in experimental example 4. For these experiments, EVs derived from celery (E1) and kiwifruit (E2) were loaded with mRNA molecules coding for the nucleocapsid (N) protein of SARS-CoV-2. The amount of preserved mRNA into the EVs after lyophilization and storage at +4° C. for 7 days was measured by qRT-PCR assay and expressed as percentage relative to the starting amount. N=3 experiments were performed for each data set. Data are shown as mean±standard deviation (SD).

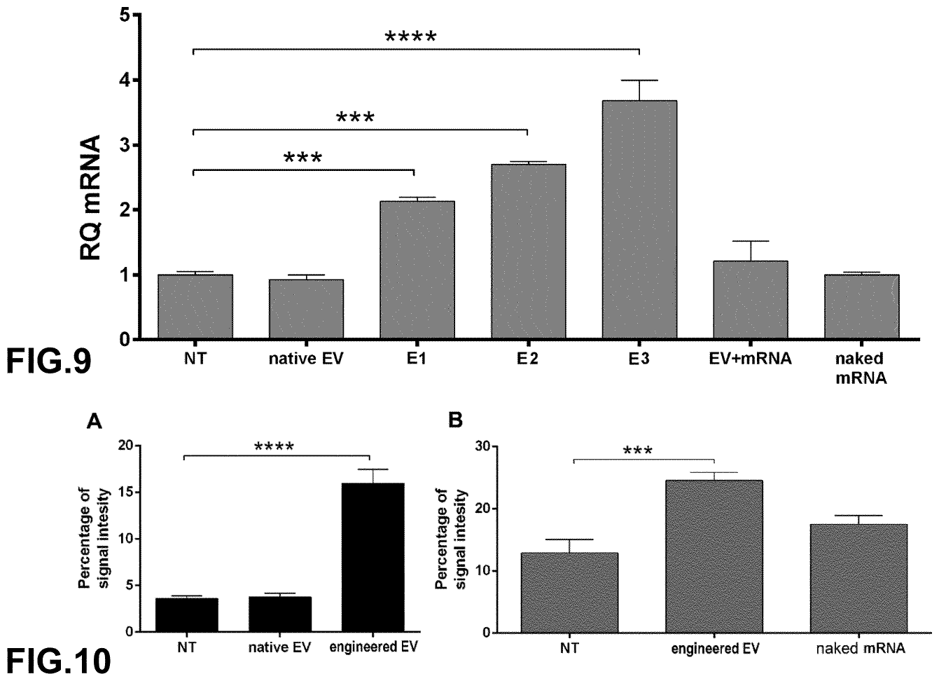

FIG. 9 shows the transfer of the nucleic acid molecules loaded into the engineered plant-derived EVs of the invention to recipient cells in experimental example 5. EVs of the invention loaded with mRNA molecules coding for the nucleocapsid (N) protein of SARS-CoV-2were incubated with macrophages. After 24 hours, the amount of mRNA was measured in recipient cells using molecular analysis (qRT-PCR), normalized to GAPDH as housekeeping and expressed as RQ value as described in the method section. The RQ values were normalized to the control (untreated cells, NT) and a RQ value of 1 means that the mRNA is not detectable in the sample. Macrophages were treated with native plant-derived EVs (native EV), engineered plant-derived EVs (E1, E2, E3), plant-derived EVs incubated with mRNA (EV+mRNA) without nucleic acid loading, naked mRNA. Recipient cells were treated with a dose of 50,000 particles/cell. The experiment was performed with EVs from cabbage (E1), pomegranate (E2) and kiwifruit (E3). The statistical significance was calculated comparing the RQ value of the mRNA for each sample with untreated cells as control (NT). p: *** <0.005, **** <0.001. N=3 experiments were performed for each data set. Data are shown as mean±standard deviation (SD).

FIG. 10 shows the functionality of nucleic acid molecules carried by engineered plant-derived EVs of the invention in recipient cells in experimental example 5. For these experiments, the EVs of the invention loaded with mRNA molecules coding for the green fluorescent protein (GFP) were incubated with (A) endothelial cells and (B) macrophages as recipient cells. After 24 hours of co-incubation, the expression of the protein encoded by the exogenous mRNA in recipient cells was detected as fluorescent signal using cytofluorimetric analysis (FACS). Recipient cells were treated with native plant-derived EVs (native EV), engineered plant-derived EVs of the invention or naked mRNA at a dose of 50,000 particle/cell. The experiments were performed with plant-derived EVs from courgette (FIG. 10A) and kale (FIG. 10B). The statistical significance was calculated comparing the percentage of signal intensity for each sample with untreated cells as control (NT). p: *** <0.005, **** <0.001. N=3 experiments were performed for each data set. Data are shown as mean±standard deviation (SD).

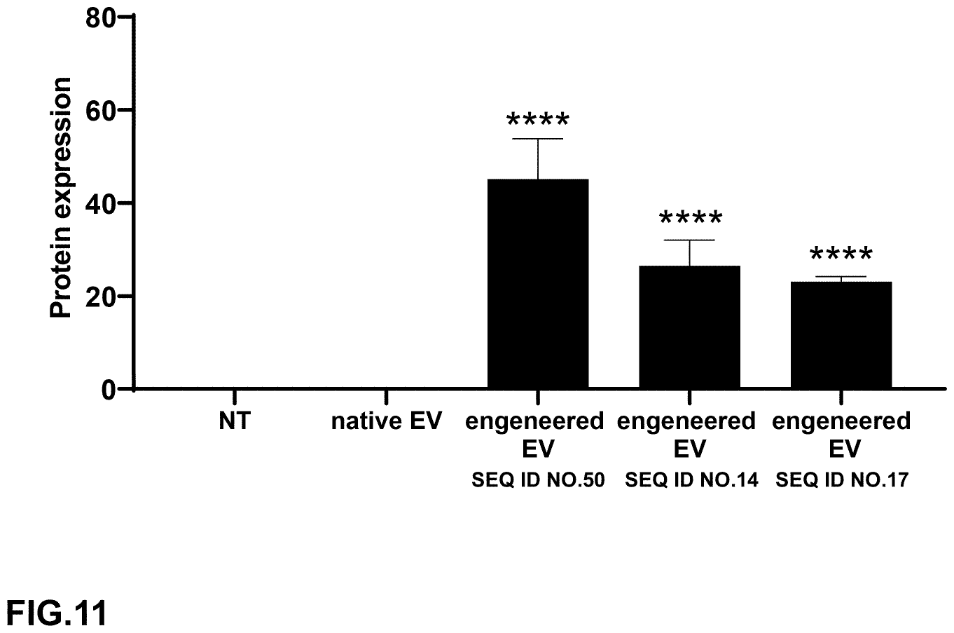

FIG. 11 shows the protein expression in target recipient cells treated with nucleic acid molecules carried by engineered plant-derived EVs of the invention in experimental example 5. For these experiments, the EVs of the invention loaded with mRNA molecules coding for the SARS-CoV-2 Spike Glycoprotein (S1) RBD protein (SEQ ID NO. 50), SARS-CoV-2 Spike Glycoprotein full protein (SEQ ID NO. 14), or SARS-CoV-2 Nucleocapsid Protein (SEQ ID NO. 17) were incubated with endothelial cells as recipient cells. After 24 hours of co-incubation, the expression of the protein encoded by the exogenous mRNA in recipient cells was detected using fluorescent-labelled secondary antibody and the fluorescent signal was measured with cytofluorimetric analysis (FACS). Recipient cells were treated with native plant-derived EVs (native EV) or engineered plant-derived EVs of the invention at a dose of 1.2×10¹⁰ particles. The experiments were performed with orange-derived EVs. The statistical significance was calculated comparing the percentage of signal intensity for each sample with untreated cells as control (NT). p: **** <0.001. N=3 experiments were performed for each data set. Data are shown as mean±standard deviation (SD).

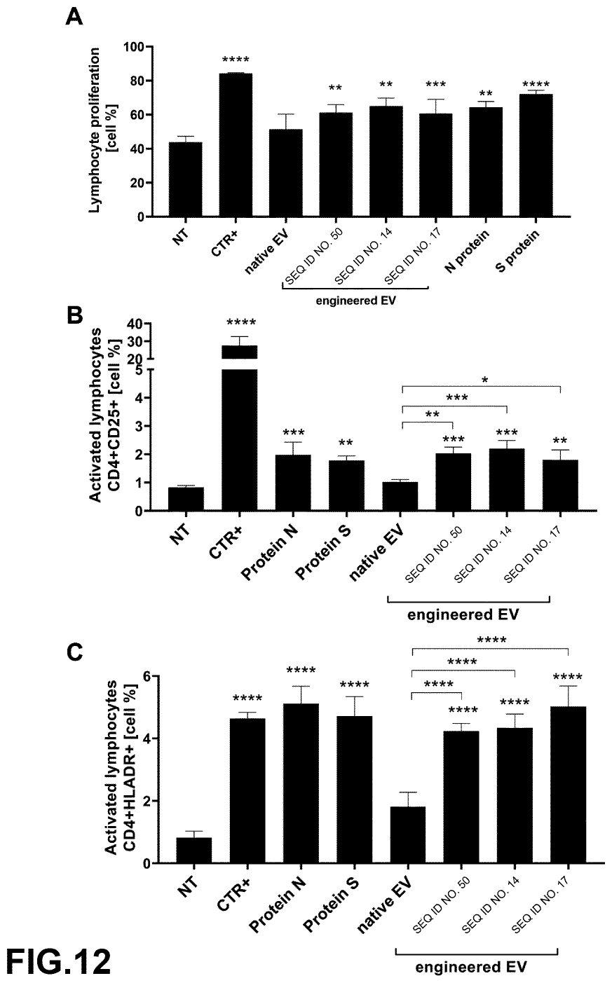

FIG. 12 shows that plant-derived EVs of the invention engineered with nucleic acids, and not native EVs, are able to activate lymphocytes after incorporation into macrophages in experimental example 6. For these experiments, the EVs of the invention loaded with mRNA molecules coding for the SARS-CoV-2 Spike Glycoprotein (S1) RBD protein (SEQ ID NO. 50), SARS-CoV-2 Spike Glycoprotein full protein (SEQ ID NO. 14), or SARS-CoV-2 Nucleocapsid Protein (SEQ ID NO. 17) were incubated with APC cells (macrophages) as recipient cells. After EV incorporation in APCs, PBMC cells were added and the treatment with engineered EVs was repeated every five days for two times. At the end, lymphocytes were analyzed by cytofluorimetric analysis (FACS). Lymphocytes, identified by the expression of CD4+, were evaluated for their activation. Lymphocyte activation was measured as increase of lymphocyte proliferation (A) and increase of the expression of lymphocyte activation markers CD25+ (B) and HLADR+ (C) of lymphocytes CD4+. Cells were treated with native plant-derived EVs (native EV) or engineered plant-derived EVs of the invention at a dose of 1.2×10¹⁰ particles. The experiments were performed with orange-derived EVs. The statistical significance was calculated comparing the percentage of signal intensity for each sample with untreated cells as control (NT) or cells treated with native EVs. Positive controls were represented by treatment with beads human T-Activator CD3/CD28 (CTR+) and purified proteins (SARS-CoV-2 Spike Glycoprotein RBD protein (S protein), or SARS-CoV-2 Nucleocapsid protein (N protein)). p: * <0.05, ** <0.01, *** <0.005, **** <0.001. N=3 experiments were performed for each data set. Data are shown as mean±standard deviation (SD).

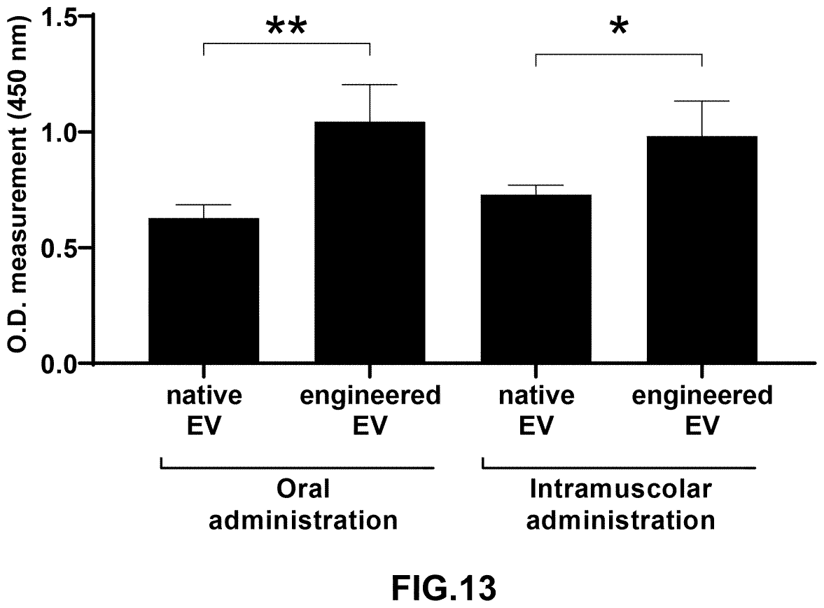

FIG. 13 shows that plant-derived EVs of the invention engineered with nucleic acids, and not native EVs, are able to induce specific immune response in mice in experimental example 7. The graph shows the absorbance measurement of IgA immunoglobulins specific for SARS-CoV-2 Spike Glycoprotein (S1) RBD protein induced by vaccination. For this experiment, mice were immunized at day 0 and day 21 and serum was analyzed at day 35 following sacrifice. Mice were treated with native EVs (native EV) or EV engineered with mRNA molecules coding for the SARS-CoV-2 Spike Glycoprotein RBD (S1). The treatment was administered via intramuscular or oral routes. The humoral immune response as IgA induction was assessed by ELISA using ELISA plates coated with the SARS-CoV-2 Spike Glycoprotein RBD (S1). The statistical significance was calculated comparing the signal intensity of native and engineered EVs for each administration route. p: * <0.05, ** <0.01 N=3 animals for each data set. Data are shown as mean±standard deviation (SD).

### MATERIALS AND METHODS

### Extracellular Vesicles Isolation

Extracellular vesicles were isolated from fresh fruit juice (kiwifruit, pomegranate, blueberry, orange, lemon) or fresh plant extract (courgette, cabbage, kale, celery). The juice or extract was sequentially filtered using decreasing order of pores to remove fibers. EV were then purified with differential ultracentrifugation or tangential flow filtration. For differential ultracentrifugation, the juice was centrifuged at 1,500 g for 30 minutes to remove debris and other contaminants. Then, EV were purified by ultracentrifugation at 10,000 g followed by ultracentrifugation at 100,000 g for 1 hour at 4° C. (Beck-man Coulter Optima L-90K). The final pellet was resuspended with phosphate buffered saline added with 1% DMSO and filtered with 0.22 micrometer filters to sterilize.

Extracellular vesicles were used or stored at −80° C. for long time. For tangential flow filtration, at first the juice was clarified by filtration with depth filter sheet discs Supracap 50 (Pall) to exclude fibers and debris. Then, the filtered juice was purified by concentration and diafiltration using a tangential flow filtration cassette TFF Omega (Pall Cadence). Finally, the retentate from tangential flow filtration was sterilized by filtration with a 0.2 nm filter.

### Nanoparticle Tracking Analysis (NTA)

Nanoparticle tracking analysis (NTA) was used to define the EV dimension and profile using the NanoSight LM10 system (Malvern), equipped with a 405 nm laser and with the NTA 3.1 analytic software. The Brownian movements of EV present in the sample subjected to a laser light source were recorded by a camera and converted into size and concentration parameters by NTA through the Stokes-Einstein equation. Camera levels were for all the acquisition at 16 and for each sample, three videos of 30 s duration were recorded. Briefly, purified EVs were diluted 1:2000 in 1 ml vesicle-free saline solution (Fresenius Kabi). NTA post-acquisition settings were optimized and maintained constant among all samples, and each video was then analyzed to measure EV mean, mode and concentration.

### Production of the EVs of the Invention

The engineered plant-derived EVs of the invention were produced by sequential steps as described as follows. Briefly, plant-derived EVs were mixed with a cationic peptide and the reaction was carried out at 37° C. for 1 hour. A preparation of nucleic acid molecules was mixed with a sugar and the reaction was carried out at 20° C. for 10 minutes. Then, the two solutions were mixed and the reaction was carried out at 37° C. for 3 hours. Then, water was added to the reaction and samples were put at 4° C. for 12 hours. In order to purify the engineered plant-derived EVs from remaining free nucleic acid molecules, samples were washed by ultracentrifugation at 100,000 g for 2 hours at 4° C. (Beckman Coulter Optima L-90K, Fullerton, CA, USA) and samples were resuspended in saline solution.

### Measurement of EVs Membrane Potential

The analysis was performed by using the Zeta-sizer nanoinstrument (Malvern Instruments, Malvern, UK). All samples were analyzed at 25° C. in filtered (cutoff=200 nm) saline solution. Zeta-potential (slipping plane) was generated at x distance from the vesicle indicating the degree of electrostatic repulsion between adjacent, similarly charged vesicles in a dispersion.

### Protein Extraction and Quantification

Proteins were extracted from EVs samples using RIPA buffer (150 nM NaCl, 20 nM Tris-HCl, 0.1% sodium dodecyl sulfate, 1% deoxycholate, 1% Triton X-100, pH 7.8) supplemented with a cocktail of protease and phosphatase inhibitors (Sigma-Aldrich, St. Louis, Missouri, USA). The protein content was quantified by BCA Protein Assay Kit (Thermo Fisher Scientific, Waltham, Massachusetts, USA) following manufacturer's protocol. Briefly, 10 μl of sample were dispensed into wells of a 96-well plate and total protein concentrations were determined using a linear standard curve established with bovine serum albumin (BSA).

### Phosphatidylserine Analysis

For phosphatidylserine analysis, EVs samples were stained with Annexin V FITC and FITC isotype (Miltenyi Biotec, Germany) for 30 minutes and diluted with saline solution before acquisition. Samples were characterized by cytofluorimetric analysis using the CytoFLEX flow cytometer (Beckman Coulter) with CytExpert software and the percentage of signal positivity was measured for each sample using FITC isotype as background.

### RNA Extraction and Quantification

Total RNA was isolated from EVs and cells using the miRNeasy Mini Kit (Qiagen, Hilden, Germany) according to the manufacturer's protocol and resuspended in water. RNA concentration of samples was quantified using spectrophotometer (mySPEC, VWR, Radnor, PA, USA).

### mRNA Detection Using qRT-PCR

From RNA samples, cDNA was obtained using High-Capacity cDNA Reverse Transcription Kit (Applied Biosystems). Five nanograms of cDNA were added to SYBR GREEN PCR Master Mix (Applied Biosystems) and run on a 96-well QuantStudio12K Flex Real-Time PCR (qRT-PCR) system (Thermo Fisher Scientific, Waltham, MA, USA). GAPDH was used as a housekeeping gene in cell samples. Fold change (Rq) in mRNA expression among all samples was calculated as 2-ΔΔCt respect control samples.

### Cell Cultures

Human microvascular endothelial cells (HMEC) were obtained by immortalization with simian virus 40 of primary human dermal microvascular endothelial cells. HMEC were cultured in Endothelial Basal Medium supplemented with bullet kit (EBM, Lonza, Basel, Switzerland) and 1 ml Mycozap CL (Lonza). Macrophage MV-4-11 cell line (ATCC® CRL9591™) was obtained by ATCC and cultured in Iscove's Modified Dulbecco's Medium supplemented with 10% of fetal bovine serum (ATCC, USA). Peripheral blood mononuclear cells (PBMC) were isolated as follow: whole blood from healthy volunteer donors was diluted 1:1 with PBS, then 30 ml were gently layered above 15 ml of Histopaque (Sigma-Aldrich) in a 50 ml centrifuge tube. The tube was centrifuged for 30 minutes at 400 g. The white and cloudy layer containing PBMC was collected in a 50 ml centrifuge tube, diluted with 40 ml of PBS and centrifuged 5 minutes at 300 g for washing, for two times. The pelleted cells were counted, and the percentage viability estimated using Trypan blue staining. Cells were cultured in RPMI with 10% fetal bovine serum in 24 well plates.

### Nucleic Acid Incorporation in Recipient Cells

In order to evaluate the uptake into cells of eGFP mRNA loaded into engineered plant-derived EVs of the invention, these vesicles were incubated with HMEC cells and macrophages. A total of 50,000 recipient cells/well were plated in 24-well plates and stimulated with 50,000 vesicles/recipient cell. After 24 hours, cells were extensively washed, detached with trypsin and the fluorescence of translated GFP protein was measured by FACS using the CytoFLEX flow cytometer with CytExpert software (Beckman Coulter Optima L-90K, Fullerton, CA, USA).

### In Vitro Nucleic Acid Degradation Assay

In order to test the resistance to enzyme degradation of nucleic acid molecules loaded into the EVs of the invention, the inventors carried out a RNAse assay. Briefly, samples were treated with RNase A (ThermoFisher Scientific), using a concentration of 0.4 mg/mL, for 30 min at 37° C. The RNase inhibitor (Thermo Fisher Scientific) was used to stop the reaction as described by the manufacturer's protocol, and samples were washed by ultracentrifugation at 100,000 g for 2 h at 4° C. using a 10 mL polycarbonate tube (SW 90 Ti rotor, Beckman Coulter Optima L-90 K ultracentrifuge). Eventually, samples of EV pellets were resuspended in saline buffer solution and molecular analysis was performed.

In order to test resistance to stomach digestion of nucleic acid molecules loaded into the EVs of the invention, the inventors carried out a stomach digestion assay. Briefly, a stomach-like solution was prepared containing 18.5% w/v HCl (pH 2.0), 24 mg/mL of bile extract, pepsin solution (80 mg/mL in 0.1 N of HCl, pH 2.0; Sigma) and 4 mg/mL of pancreatin (Sigma) in 0.1 N of NaHCO3. An amount of 1 μl of each EVs sample in a water solution was incubated with slow rotation at 37° C. for 60 min with 1.34 μL of stomach-like solution. The pH value of the stomach-like solution was adjusted to 6.5 with 1 N NaHCO₃ and was referred to as an intestinal solution. Then, EV samples were incubated for additional 60 min in the intestinal solution. The stability of the nucleic acid molecules loaded into the EVs of the invention was evaluated by molecular analysis as above described. For all resistance experiments, naked RNA was used as control.

### Sample Lyophilization

Samples were lyophilized using the instrument Heto lyolab 3000 (Thermo Fisher Scientific) for 3 hours and kept for 7 days at 4° C. After storage time point, EV samples were analyzed for their content of nucleic acid using molecular analysis. The content thus measured was compared to the starting amount, before lyophilization and storage.

### Immune Cells Activation Assay

To assess PBMC proliferation by flow cytometric analysis, PBMC were stained with CFSE dye from CellTrace Cell Proliferation Kits (Invitrogen, ThermoFisher Scientific) according to manufacturer's instruction. PBMCs were then plated in a 48 well plate at the density of 50,000 cells/well. In order to evaluate whether the engineered, plant-derived EVs of the invention may affect PBMC proliferation, PBMCs were stimulated with these vesicles at the dose of 50,000 particles/cell. Unstimulated PBMCs were used as control. After 24 hours incubation, PBMCs were collected and fluorescence was measured by the CytoFLEX flow cytometer equipped with CytoExpert software (Beckman Coulter). CFSE dye is detected as FITC fluorescence.

To analyse PBMC proliferation by Bromo Deoxyuridine (BrdU) incorporation assay, PBMCs were plated in a 96 well plate at the density of 20,000 cells/well and 10 μl of BrdU labeling solution (BrdU colorimetric assay, Roche) were added to each well. In order to evaluate whether the engineered, plant-derived EVs of the invention may affect PBMC proliferation, PBMCs were stimulated with these vesicles at the dose of 50,000 particles/cell. Un-stimulated PBMCs were used as control. Moreover, to assess whether the EVs of the invention may reduce proliferation of activated PBMC, PBMCs were stimulated with these vesicles at the dose of 50,000 particles/cell and LPS (from E.coli, Sigma-Aldrich) at the concentration of 100 ng/ml. Unstimulated PBMCs were used as negative control. The effects of the stimuli were analyzed after 48 hours of incubation. The assay was carried out according to manufacturer's instructions. Absorbance was measured by an ELISA reader at 420 nm with the reference wavelength at 490 nm. The mean absorbance for each condition was calculated. Absorbance is directly proportional to proliferation rate.

### Detection of Protein Expression

To assess the protein expression in target cells, endothelial cells were stimulated with 1.2×10¹⁰ EVs. The assayed cell samples included untreated cells (NT), cells treated with plant-derived native EVs and cells treated with plant-derived EVs of the invention engineered with mRNA molecules coding for the SARS-CoV-2 Spike Glycoprotein (S1) RBD protein (SEQ ID NO. 50), SARS-CoV-2 Spike Glycoprotein full protein (SEQ ID NO. 14), or SARS-CoV-2 Nucleocapsid Protein (SEQ ID NO. 17). After 24 hours, cells were extensively washed, detached with trypsin and fixed and permeabilized following manufacturer's instruction (Inside Stain Kit, Miltenyi Biotec). Then, cells were stained for 30 minutes at room temperature with specific antibody to detect protein expression (antibody against SARS-CoV-2 Spike Glycoprotein and Nucleocapsid protein, Invitrogen). Following a washing, fluorescent secondary antibodies were added for 1 hour at room temperature (Alexa Fluor Plus 594 or 488, Invitrogen, ThermoFisher Scientific). After a wash, cells were resuspended in appropriate buffer and acquired by FACS using the CytoFLEX flow cytometer with CytExpert software (Beckman Coulter Optima L-90K, Fullerton, CA, USA).

### Lymphocyte Activation Assay

To assess the ability of engineered EVs of the invention to induce lymphocyte activation following incorporation into APCs, macrophages were plated 20.000 cells/well in a 24 well plate and stimulated with 1.2×10¹⁰ EVs. The assayed cell samples included untreated cells (NT), cells treated with plant-derived native EVs and cells treated with plant-derived EVs of the invention engineered with mRNA molecules coding for the SARS-CoV-2 Spike Glycoprotein (S1) RBD protein (SEQ ID NO. 50), SARS-CoV-2 Spike Glycoprotein full protein (SEQ ID NO. 14), or SARS-CoV-2 Nucleocapsid Protein (SEQ ID NO. 17). After incorporation, PBMC were added at the concentration of 200,000 cells/well and the treatment with EVs was repeated after 5 days. After 10 days from the initial treatment, cells were harvested and stained with fluorescent antibody for CD4, CD25 and HLA DR using appropriate isotype (Miltenyi Biotec) for 30 minutes at room temperature. Following a wash, cells were acquired.

For proliferation analysis, the employed PBMCs were previously stained with CSFE dye from CellTrace Cell Proliferation Kits (Invitrogen, ThermoFisher Scientific) according to manufacturer's instruction.

Finally, samples were analyzed by FACS using the CytoFLEX flow cytometer with CytExpert software (Beckman Coulter Optima L-90K, Fullerton, CA, USA).

### Mice Vaccination

Female BALB/cAnNCrl mice, 6-10 weeks old, received 2 immunizations at day 0 and day 21 with a dose of engineered plant-derived EVs of the invention equivalent to 30 μg of mRNA and were sacrificed at day 35. Mice were treated with plant-derived native EVs or plant-derived EVs of the invention engineered with mRNA molecules coding for the SARS-CoV-2 Spike Glycoprotein RBD (S1) using oral (using gavage) and intramuscular (right leg) routes. After the sacrifice, blood was collected to isolated sera for antibody detection.

### Antibody Measurement

SARS-CoV-2 specific IgA antibody titers of sera were determined by ELISA. Briefly, MaxiSorp ELISA plates (Nunc) were coated with 1 μg/ml of SARS-CoV-2 Spike protein (Thermofisher Scientific) in 100 μl of 50 mM sodium carbonate/bicarbonate pH 9.6 buffer per well, overnight at 4° C. Coated plates were washed 3 times with 200 μl of 1× PBS and saturated with 200 μl 3% BSA in 1× PBS per well. Plates were washed three times with 1× PBS, and incubated in 3% BSA and with 100-fold mouse sera dilution for 2 h. This was followed by 3 washes with 200 μl of 1× PBS per well and incubation with 100 μl per well of secondary donkey anti-mouse IgA HRP conjugated antibody diluted 1:10,000. Following the incubation with the secondary antibody, plates were washed 5 times with 200 μl of 1× PBS per well and developed with 100 μl of TMB per well (Thermofisher Scientific) for 30 minutes. The reaction was stopped by adding 100 μl of stop solution (Thermofisher Scientific) per well. The 450 nm absorbance was read using plate reader.

### Statistical Analysis

Data analysis was carried out with the software Graph Pad 8, demo version. Results are expressed as mean±standard deviation (SD). One-way analysis of variance (ANOVA) was used to substantiate statistical differences between groups, while Student's t-test was used for comparison between two samples. We used p<0.05 as a minimal level of significance.

### RESULTS/EXAMPLES

### Example 1

To investigate the feasibility of the method of the present invention, the inventors engineered different plant-derived EVs and characterized them according to a number of features traditionally used to characterize EVs in terms of physical properties, such as particle size and surface charge (Théry C., et al, (2018) “Minimal information for studies of extracellular vesicles 2018 (MISEV2018): a position statement of the International Society for Extracellular Vesicles and update of the MISEV2014 guidelines”; Journal of Extracellular Vesicles, 7:1, 1535750, DOI: 10.1080/20013078.2018.1535750). FIG. 1 shows the size of engineered, plant-derived EVs of the invention from different plants, including kiwifruit, cabbage, and blueberry. Similar results were also obtained with EVs from other plant sources such as pomegranate, kale, celery, courgette, orange, and lemon. Particle size is a fundamental parameter of EVs. Data obtained showed that the EVs of the invention have a higher mean diameter compared to native plant-derived EVs (FIG. 1A, B, C). The measurement of EV size was carried out by the inventors with Nanoparticle tracking Analysis (NTA) technique, the most used method to measure EV size which utilizes the properties of both light scattering and Brownian motion in order to obtain the nanoparticle size distribution of samples in liquid suspension. In particular, NTA works by tracking particle motion via light scattering to assess the mean squared displacement of particles moving under Brownian motion, in a sample chamber illuminated by a laser beam. The tracking of particles enables a diffusion constant to be calculated, which is used in the Stokes-Einstein equation to calculate hydrodynamic diameters. The Stokes-Einstein equation also takes into account the temperature and viscosity of the suspension. The results of the analysis performed by the inventors demonstrate that the EVs of the invention have a higher size compared to native plant-derived EVs as the native vesicles have a diameter ranging from 100 to 150 nm, with a mean diameter of 134±6 nm, whereas the diameters of the EVs of the invention were in the range between 200 and 250 nm, with a mean diameter of 220 nm. The size distribution demonstrated that EVs of the invention have a diameter ranging from 20 to 500 nm, preferably ranging from 200 to 300 nm.

In order to characterize the membrane properties of engineered, plant-derived EVs of the invention, vesicle membrane potential was measured. In fact, Zeta potential is a popular method to measure the surface potential of EVs and it is used as an indicator of surface charge and colloidal stability. The surface charge of EVs depends on the nature of molecules expressed at their surfaces and it affects EV interaction in dispersed systems such as human body, defining their activity in biological processes. For example, the surface charge is known to influence different biological processes associated with particles, such as cellular uptake and cytotoxicity. Zeta potential is a measure of the magnitude of the electrostatic or charge repulsion/attraction between particles and it can be measured from the electrophoretic mobility in a suspension determined by applying an electric field and measuring the resulting velocity of the particles (Electrophoretic light scattering) (Midekessa G, et al. Zeta Potential of Extracellular Vesicles: Toward Understanding the Attributes that Determine Colloidal Stability. ACS Omega. 2020 Jun. 30;5(27):16701-16710. doi: 10.1021/acsomega.0c01582. PMID: 32685837; PMCID: PMC7364712.) As shown in FIG. 2, plant-derived EVs are known to have a negative surface charge and the membrane potential (Z potential) was ranging between −10 and −15 mVolt, with a mean value of −13 mVolt. Instead, engineered plant-derived EV of the invention exploited a different membrane with a membrane potential comprises between 0 and −3 mVolt, with a mean value of −2 mVolt. The above described experiment was performed on engineered, plant-derived EVs from courgette (E1) and blueberry (E2), but similar results were obtained with EVs derived from other plant sources such as kiwifruit, cabbage, kale, pomegranate, lemon, orange and celery.

To further characterize the EVs of the invention, the protein content of these vesicles and native plant-derived EVs was measured (FIG. 3A). The data thus obtained demonstrated that the EVs of the invention have a higher protein content ranging from 120 to 160 ng/10¹⁰ EVs, whereas native plant-derived EVs have a protein content ranging from 50 to 100 ng/10¹⁰ EVs. The inventors carried out the experiments on engineered plant-derived EVs from pomegranate (E1) and kiwifruit (E2), but similar results were obtained with EVs derived from other plant sources such as courgette, cabbage, kale, blueberry, lemon, orange and celery.

Moreover, the present inventors carried out dedicated experiments to better characterize the membrane of the EVs of the invention. As is known in the art, in native EVs phosphatidylserine (PS) is predominantly located along the outer surface of the plasma membrane. Without wishing to be bound by any theory, the inventors believe that, upon membrane rearrangement due to the osmotic stress, PS loses its asymmetric distribution in the phospholipid bilayer and translocates to the inner side of the membrane in the EVs of the invention. The detection of phosphatidylserine in the extracellular membrane of vesicles was achieved by means of fluorescently labeled Annexin V. In fact, Annexin V is known to specifically bind to PS on vesicle membrane. The quantity of fluorescent signal of Annexin V reflects the PS content on the outer surface of the EV membrane (Montoro-García S, et al. “An innovative flow cytometric approach for small-size platelet microparticles: influence of calcium”. Thromb Haemost. 2012 August;108(2):373-83). The results obtained by the present inventors showed that a percentage ≤44% of EVs in the composition of the invention have phosphatidylserine in the outer layer of the membrane (FIG. 3B), such percentage ranging from 40 to 44%, with a mean value of 43%. Differently, in a composition comprising native plant-derived EVs, the percentage of these vesicles having phosphatidylserine in the outer layer of the membrane ranges from 55 to 48%, with a mean value of 49%.

The experiments above described were performed on engineered, plant-derived EVs from cabbage (E1) and blueberry (E2), but similar results were also obtained with EVs from other plant sources such as kiwifruit, lemon, orange, courgetti, kale, pomegranate and celery.

As a further assessment, the present inventors conducted dedicated experiments with the aim of evaluating the immunomodulatory activity of the engineered, plant-derived EVs of the invention. Briefly, PBMC, i.e. a mixed population of lymphocytes, monocytes and other immune cells from the human blood, were stimulated with the EVs of the invention and cell proliferation rate was measured.

As shown in FIG. 4A, the proliferation rate of PBMC stimulated for 48 hours with the EVs of the invention from pomegranate is the same as untreated PBMC, suggesting that these vesicles do not promote PBMC proliferation and do not exert an immunostimulatory effect. Further, to verify whether the EVs of the invention have an immunosuppressive effect, the present inventors treated PBMC with LPS, which is known to induce inflammatory responses and promote lymphocyte proliferation, and then stimulated the cells with the engineered, plant-derived EVs of the invention. As shown in FIG. 4B, the proliferation rate of PBMC activated by LPS was not affected by the EVs of the invention (from pomegranate (E1) and kiwifruit (E2)).

These results confirm that the EVs according to the invention do not have immunostimulating nor immunosuppressive effects.

Furthermore, PBMC were also stained with a fluorescent dye (CFSE) which allows the detection of PBMC proliferation by flow cytometry. As shown in FIGS. 4C and 4D, proliferation rate of PBMC stimulated with engineered, plant-derived EVs from cabbage (E1), celery (E2), and courgettes (E3) is the same as unstimulated PBMC. Similar results were obtained with engineered EVs from other plants, such as blueberry, lemon, orange, and kale.

Overall, the above results demonstrate that engineered, plant-derived EVs of the invention are not able to stimulate nor suppress immune cell activation and proliferation, and confirm the non-immunomodulating properties of these vesicles regardless of vesicles plant source.

Finally, the total RNA content of EVs of the invention and native plant-derived EVs was measured (FIG. 5). The data obtained demonstrated that the EVs of the invention have a higher RNA content compared to native vesicles, ranging from 30 to 100 ng/10⁹ EVs, with a mean value of 50 ng/10⁹ EVs. Native plant-derived EVs have an RNA content ranging from 5 to 15 ng/10⁹ EVs, with a mean value of 10 ng/10⁹ EVs. The experiments were performed on engineered EVs from celery (E1), pomegranate (E2) and kiwifruit (E3), but similar results were also obtained with EVs from other plant sources such as courgette, cabbage, kale, lemon, orange and blueberry.

Taken together, all these data demonstrate that engineered, plant-derived EVs of the invention differ significantly from native plant-derived EVs. In particular, upon exogenous nucleic acid loading, unique alterations surprisingly occur in the plant-derived EVs structure and function compared to the native vesicles, thereby resulting in higher mean diameter, higher surface charge, lower phosphatidylserine content and loss of immunomodulatory effect on immune system cells.

### Example 2

With the aim of demonstrating the suitability of the EVs according to the invention as vehicles for nucleic acid delivery, engineered EVs were produced by internally loading mRNA molecules, and the amount of loaded mRNA was measured by qRT-PCR analysis (FIG. 6). The results obtained showed that the EVs according to the invention can be loaded with increasing doses of nucleic acid molecules. In fact, engineered EVs were produced from kiwifruit (E1 and E3) and celery (E2 and E4) using two different doses of mRNA: 0.1 μg/ml for E1 and E2, and 1 μg/ml for E3 and E4. The nucleic acid dose increase was detectable as increase of the amount of mRNA in vesicles (E3 and E4 versus E1 and E2, respectively). Similar results were also obtained with EVs from other plant sources such as courgette, cabbage, kale, lemon, orange, pomegranate and blueberry. Taken together, these data demonstrated that EVs of the invention encapsulate the loaded nucleic acid molecules and their amount can be increased.

### Example 3

The present inventors conducted dedicated experiments to assess the capacity by the EVs of the invention to preserve loaded nucleic acid molecules from degradation (FIG. 7). In particular, these studies showed that engineered, plant-derived EVs of the invention were able to protect loaded nucleic acid molecules from the treatment with degrading enzyme (RNAse). Briefly, following EVs treatment with RNAse, qRT-PCR analysis revealed that about 80% of the loaded mRNA was still present in the vesicles of the invention, whereas naked mRNA used as control was almost completely degraded (FIG. 7A)

Further experiments showed that the EVs according to the invention are also able to protect loaded nucleic acid molecules since, upon vesicles treatment with a stomach-like solution mimicking the gastrointestinal environment, about 90% of the loaded mRNA was still present in the EVs whereas the naked mRNA was almost completely degraded (FIG. 7B). The above experiments were performed on engineered, plant-derived EVs from pomegranate (6A) and kiwifruit (6B), but similar results were also obtained with EVs from other plant sources such as courgette, cabbage, kale, blueberry, lemon, orange and celery.

Taken together, these data demonstrate that the EVs according to the invention protect the encapsulated exogenous nucleic acid from degrading conditions. Moreover, the protection from gastrointestinal environment supports the oral administration of the composition for use according to the invention.

### Example 4

Engineered, plant-derived EVs according to the invention can be efficiently lyophilized and stored. In particular, following EVs lyophilization and storage at +4° C. for 7 days, the content of loaded mRNA in the vesicles did not decrease compared to the starting condition (FIG. 8). The experiments were performed on engineered, plant-derived EVs from celery (E1) and kiwifruit (E2), but similar results were also obtained with EVs from other plant sources such as courgette, cabbage, kale, blueberry, lemon, orange and pomegranate.

These data demonstrated that the EVs according to the invention can be easily lyophilized and stored efficiently at +4° C. or at room temperature, without the need to use very low temperatures typical of storage of nucleic acid agents, such as −80° C.

### Example 5

The inventors have further demonstrated that engineered, plant-derived EVs of the invention are suitable to be used for delivering loaded nucleic acids to recipient cells (FIG. 9). In these experiments, macrophages were used as exemplary recipient cells and the transfer of mRNA molecules in these cells was measured by qRT-PCR analysis. In particular, the above experiments demonstrated that EVs of the invention derived from different types of plants (E1, E2, E3) were able to transfer the mRNA molecules to macrophages relative to untreated cells (not treated, NT), whereas no mRNA transfer was detected in native plant-derived EVs (native EV), plant-derived EVs co-incubated with the mRNA without nucleic acid loading (EV+mRNA) and naked mRNA. The experiments were performed on engineered, plant-derived EVs from cabbage (E1), pomegranate (E2) and kiwifruit (E3), but similar results were also obtained with EVs from other plant sources such as courgette, celery, kale, lemon, orange and blueberry.

In order to demonstrate that the nucleic acid delivered to recipient cells maintains its functional activity, EVs of the invention were assayed containing mRNA molecules coding for the GFP protein. After incorporation into recipient cells, the mRNA, if functional, is translated into the GFP protein and the fluorescence of the functional protein is detectable in cells.

The experiments carried out by the inventors showed that the mRNA carried by the EVs of the invention was functional and was detectable as fluorescent signal in endothelial cells (FIG. 10A) and macrophages (FIG. 10B), whereas there was no transfer of functional mRNA using native plant-derived (native EV, in FIG. 10A) or naked mRNA (naked mRNA, in FIG. 10B). The experiments were performed on engineered, plant-derived EVs from courgette (FIG. 10A) and kale (FIG. 10B), but similar results were obtained with EVs from other plant sources such as celery, cabbage, kiwifruit, blueberry, lemon, orange and pomegranate.

Furthermore, the inventors demonstrated that engineered, plant-derived EVs of the invention are able to transfer functional mRNAs which are translated into protein antigens into recipient cells and expressed as correctly folded antigen (FIG. 11). In the course of the experiments, EVs of the invention were assayed containing mRNA molecules coding for different sequences of viral protein antigens: SEQ ID NO. 50, SARS-CoV-2 Spike Glycoprotein (S1) RBD protein; SEQ ID No. 14, SARS-CoV-2 Spike Glycoprotein full protein; SEQ ID NO. 17, SARS-CoV-2 Nucleocapsid Protein. After incorporation into endothelial cells, the mRNA, if functional, is translated into the protein which can be detected by specific antibody. FIG. 11 shows that engineered, plant-derived EVs of the invention are able to transfer the mRNA into target cells which translate it into the specific protein antigen, whereas no protein antigens were detectable in untreated cells (NT) or cells treated with native plant-derived, not engineered, EVs.

Taken together, these data demonstrated that the EVs according to the invention can efficiently deliver exogenous nucleic acid molecules to different types of recipient cells, including antigen presenting cells (APC) as macrophages, preserving at the same time the nucleic acid function and its ability to be translated into protein. Thus, the correctly folded expressed protein can function as antigen in promoting the immunization by the APC. Moreover, the experiments performed as above described showed that the EVs of the invention are suitable to be used with nucleic acid molecules encoding different proteins such as viral antigens (the nucleocapsid (N) protein and Spike Glycoprotein of SARS-CoV-2) or other proteins such as GFP.

### Example 6

The inventors have further demonstrated that engineered, plant-derived EVs of the invention can deliver nucleic acids to APC cells which express and present the antigen and then stimulate specific immune activation (FIG. 12). In these experiments, macrophages were used as exemplary APC recipient cells and were stimulated with engineered EVs of the present invention before the incubation with PBMC, i.e. a mixed population of lymphocytes, monocytes and other immune cells from the human blood. For the experiments, EVs of the invention were assayed containing mRNA molecules coding for different sequences of viral protein antigens used as example: sequence 1, SARS-CoV-2 Spike Glycoprotein (S1) RBD protein; sequence 2, SARS-CoV-2 Spike Glycoprotein full protein; sequence 3, SARS-CoV-2Nucleocapsid Protein. The activation of lymphocytes was measured after ten days using FACS analysis as increase of lymphocyte (cell CD4+) proliferation and expression of activation markers CD25 and HLA DR. In order to detect lymphocytes proliferation, PBMC were also stained with a fluorescent dye (CFSE) which allows the detection of PBMC proliferation by flow cytometry. As shown in FIG. 12A, proliferation rate of lymphocyte CD4+ stimulated with engineered, plant-derived EVs is increased in comparison to negative controls, untreated cells (NT) and cells treated with native EVs. As expected, positive controls stimulated lymphocyte proliferation: human T-Activator CD3/CD28 (CTR+) and purified proteins (SARS-CoV-2 Spike Glycoprotein RBD protein (S protein), or SARS-CoV-2Nucleocapsid protein (N protein)). The increased proliferation rate of lymphocytes demonstrates their activation.

Furthermore, the stimulation of APC with engineered, plant-derived EVs of the invention increased the expression of activation markers CD25 (FIG. 12B) and HLA DR in lymphocytes (FIG. 12C).

The stimulation with the plant-derived EVs of the invention induced an increased expression of both activation markers by CD4+ lymphocytes, demonstrating immune cell activation.

The stimulation was compared to negative controls, untreated cells (NT) and cells treated with native plant-derived EVs. As expected, positive controls stimulated lymphocyte proliferation: human T-Activator CD3/CD28 (CTR+) and purified proteins (SARS-CoV-2 Spike Glycoprotein RBD protein (S protein), or SARS-CoV-2 Nucleocapsid protein (N protein)).

Overall, the above results demonstrate that engineered, plant-derived EVs of the invention activate immune response following incorporation into APC (such as macrophages) and they can be assayed with different nucleic acids molecules coding for different protein antigens.

Taken together, all these data demonstrate that engineered, plant-derived EVs of the invention are able to transfer a functional mRNA to APC, which in turn is translated into a correctly folded protein antigen and can specifically activate immune response. Of note, only engineered, plant-derived EVs of the invention, and not native plant-derived EVs, induced lymphocyte activation.

### Example 7

With the aim of demonstrating the suitability of the EVs according to the invention as vehicles for nucleic acid delivery for use as a vaccine, experiments on in vivo mouse model were performed.

In particular, mice were immunized two times (with a break of three weeks between the two treatments) and the presence of specific antibodies in serum was measured after two weeks following the last dose. Mice were treated with native plant-derived EVs (native EV) or plant-derived EVs of the invention engineered with mRNA molecules coding for a viral protein antigen as example, the SARS-CoV-2 Spike Glycoprotein RBD (S1) (engineered EV), using intramuscular or oral administration routes.

FIG. 13 shows the measurement of IgA antibodies specific for SARS-CoV-2 Spike Glycoprotein RBD (S1) in mice serum. The vaccination with engineered plant-derived EVs of the invention induced the production of specific antibodies in comparison to the vaccination with native plant-derived EVs following both oral and intramuscular administration. The antibody positive response detected after oral administration demonstrates the protection from gastrointestinal environment of the composition for use according to the invention.

Taken together, all these data demonstrate that engineered, plant-derived EVs of the invention are suitable for use as a vaccine because can be loaded with nucleic acids, which are transferred to APC, translated to a correctly folded antigen, activate immune response and the production of specific antibodies in vivo. The activation of immune response is specific to the antigen because EVs of the invention do not exert per se neither immunostimulatory nor immunosuppressive effects. Furthermore, engineered, plant-derived EVs of the invention can efficiently protect the nucleic acid from degradation, allowing the vaccine administration using different routes.

What is claimed is:

1. A method for treatment or prophylaxis of a disease in a subject, said method comprising administering to the subject a vaccine composition comprising non-immunomodulating, engineered, plant-derived extracellular vesicles (EVs), wherein said EVs are delimited by a lipid bilayer membrane comprising an outer lipid layer and an inner lipid layer,
wherein said EVs are internally loaded with an exogenous nucleic acid molecule encoding at least one protein antigen;
wherein said EVs have a diameter ranging from 20 to 500 nm;
wherein the membrane potential across the lipid bilayer membrane of said EVs ranges from +5 to −5 mV; and
wherein ≤44% of the EVs in the vaccine composition comprise phosphatidylserine in the outer lipid layer of the lipid bilayer membrane.

2. The method of claim 1, wherein the loaded exogenous nucleic acid molecule is selected from the group consisting of DNA, cDNA, messenger RNA (mRNA), pre-mRNA, long-chain RNA, coding RNA, single-stranded RNA, double stranded RNA, linear RNA, RNA oligonucleotide, self-replicating RNA (replicon RNA), retroviral RNA, and viral RNA (vRNA).

3. The method of claim 2, wherein the loaded exogenous nucleic acid molecule is a mRNA molecule comprising a nucleotide sequence selected from the group consisting of SEQ ID NOs: 14, 17, 19 and 50.

4. The method of claim 1, wherein the encoded at least one protein antigen is selected from the group consisting of tumor antigens, viral antigens, bacterial antigens, fungal antigens and protozoa antigens.

5. The method of claim 4, wherein the encoded at least one protein antigen is selected from the group consisting of prostate specific antigen (PSA), prostate stem cell antigen (PSCA), prostate specific membrane antigen (PSMA), (six transmembrane epithelial antigen of the prostate 1) (STEAP1), Receptor tyrosine-protein kinase erbB-2, cell surface associated mucin 1 protein (MUC1), Tyrosinase-related protein 2 (TRP-2), Proto-oncogene B-Raf, Proto-oncogene c-Kit, GTPase NRas, melanoma-associated antigen 1, melanoma-associated antigen 1 protein, NY-ESO-1 protein, Spike protein of SARS-COV-2, N protein of SARS-COV-2, M protein of SARS-COV-2, Hemagglutinin protein of influenza A viruses, Hemagglutinin protein of influenza B virus, Neuraminidase protein of influenza A viruses, Neuraminidase protein of influenza B virus, envelope protein of HIV1, envelope protein HIV2, Major Capsid Protein L1 of HPV, Minor Capsid Protein L2 of HPV, glycoprotein of Rabies lyssavirus, glycoprotein of Human Cytomegalovirus, envelope glycoproteins E1E2 of Hepatitis C virus, Fusion protein of RSV, spike glycoprotein of Zaire ebolavirus, Protein prM of Zika virus, Serine protease NS3 of Zika virus, Serine protease subunit NS2B of Zika virus, Envelope protein E of Zika virus, Capsid protein C of Zika virus, Toxoplasma gondii proteins, including dense granule protein 6, rhoptry protein 2A, rhoptry protein 18, surface antigen 1, surface antigen 2A, Toxoplasma gondii apical membrane antigen 1, SARS-CoV-2 Spike(S) RBD protein, and any combination thereof.

6. The method of claim 5, wherein the encoded at least one protein antigen comprises an amino acid sequence selected from the group consisting of SEQ ID NOs: 1-13, 15, 16, 18, and 20-49.

7. The method of claim 1, wherein the content of the loaded exogenous nucleic acid molecule in the EVs is in the range of from 20 to 200 ng/109 EVs.

8. The method of claim 1, wherein the EVs are derived from one or more plants selected from the group consisting of: the genus Citrus, including lemon and orange; genus Actinidia, including kiwifruit; genus Cucurbita, including courgette; genus Brassica, including cabbage and kale; genus Punica, including pomegranate; genus Vaccinium, including blueberry, and genus Apium, including celery.

9. The method of claim 1, wherein the vaccine composition further comprises one or more polycationic substances, said one or more polycationic substances being associated with the outer lipid layer of the lipid bilayer membrane of the EVs through electrostatic interactions.

10. The method of claim 9, wherein the one or more polycationic substances are selected from the group consisting of cationic proteins, including protamine, cationic peptides, polypeptides, polysaccharides, glycerol, polyethylene glycol (PEG), and any combination thereof.

11. The method of claims 1, wherein the EVs are additionally loaded with one or more sugar molecules, said one or more sugar molecules being associated with the exogenous nucleic acid molecule loaded into the EVs through electrostatic interactions and hydrogen bonding.

12. The method of claim 11, wherein the one or more sugar molecules are selected from the group consisting of disaccharides, sugar alcohols, polysaccharides, and any combination thereof.

13. The method of claim 1, wherein the vaccine composition is in a form suitable for oral, intranasal or parenteral administration.

14. A method for preparing a vaccine composition comprising non-immunomodulating, engineered, plant-derived extracellular vesicles (EVs), wherein said EVs are delimited by a lipid bilayer membrane comprising an outer lipid layer and an inner lipid layer,
wherein said EVs are internally loaded with an exogenous nucleic acid molecule encoding at least one protein antigen;
wherein said EVs have a diameter ranging from 20 to 500 nm;
wherein the membrane potential across the lipid bilayer membrane of said EVs ranges from +5 to −5 mV; and
wherein ≤44% of the EVs in the vaccine composition comprise phosphatidylserine in the outer lipid layer of the lipid bilayer membrane,
the method comprising the steps of:
(i) contacting and mixing a suspension of the plant-derived EVs with one or more polycationic substances to obtain a first mixture;
(ii) contacting and mixing a preparation of nucleic acid molecules with one or more sugar molecules to obtain a second mixture, said nucleic acid molecules encoding at least one protein antigen;
(iii) admixing said first mixture and said second mixture to obtain a third mixture; and
(iv) adding to said third mixture a pre-determined volume of water, wherein a ratio of said pre-determined volume of water to a volume of the third mixture is from 5:1 to 15:1.

15. The method of claim 14, further comprising concentrating the vaccine composition obtained in step (iv).

16. The method of claim 14, wherein the one or more polycationic substances are selected from the group consisting of cationic proteins, including protamine, calcitonin peptides, plectasin, lactoferrin, protamine-like proteins, such as spermine or spermidine, nucleoline, histones, cell penetrating peptides (CPPs);
cationic peptides, including histidine-rich peptides, arginine-rich peptides, lysine-rich peptides, cationic arginine-rich peptides (CARPs); polypeptides, including poly-arginine, poly-lysine, poly-histidine, histidine-rich peptides, arginine-rich peptides, lysine-rich peptides; polysaccharides, including chitosan, glycosaminoglycan such as polysulfated glycosaminoglycan (PSGAG), cationic dextrans; glycerol, polyethylene glycol (PEG), and any combination thereof, and/or wherein the one or more sugar molecules are selected from the group consisting of disaccharides, including trehalose, maltose, lactose, sucrose, cellobiose, chitobiose, kojibiose, nigerose, isomaltose, β,β-trehalose, α,β-trehalose, sophorose, laminaribiose, gentiobiose, trehalulose, turanose, maltulose, leucrose, iso-maltulose, gentiobiulose, mannobiose, melibiose, melibiulose, rutinose, rutinulose, xylobiose; sugar alcohols, including arabitol, erythritol, glycerol, HSHs, isomalt, lactitol, maltitol, mannitol, sorbitol, xylitol; polysaccharides, including starch, glycogen, galactogen, inulin, arabinoxylans, cellulose, chitin and pectin, and any combination thereof.

17. The method of claim 1, wherein said disease is an infectious disease or cancer.

18. The method of claim 10, wherein said one or more polycationic substances are present in the vaccine composition in an amount ranging from 0.001 to 2 μg/10¹⁰ EVs.

19. The method of claim 12, wherein the content of the one or more sugar molecules in the EVs is in the range of from 0.1 to 10 mg/10¹⁰ EVs.

20. The method of claim 15, wherein the vaccine composition obtained in step (iv) is concentrated by filtration.
